SEQUENCE LISTING (10 sequences)

SEQ ID NO: 1 - DNA, 20 bp, Artificial Sequence
  b-actin.1 Primer
acccaggcattgctgacagg

SEQ ID NO: 2 - DNA, 20 bp, Artificial Sequence
  b-actin.2 Primer
ggacagtgaggccaggatgg

SEQ ID NO: 3 - DNA, 20 bp, Artificial Sequence
  Ctsb.1 Primer
tgcgttcggtgaggacatag

SEQ ID NO: 4 - DNA, 18 bp, Artificial Sequence
  Ctsb.2
cgggcagttggaccattg

SEQ ID NO: 5 - DNA, 18 bp, Artificial Sequence
  Ctsl.1 Primer
gcacggcttttccatgga

SEQ ID NO: 6 - DNA, 20 bp, Artificial Sequence
  Ctsl.2 Primer
ccacctgcctgaattcctca

SEQ ID NO: 7 - DNA, 21 bp, Artificial Sequence
  Ctsx.1
tatgccagcgtcaccaggaac

SEQ ID NO: 8 - DNA, 24 bp, Artificial Sequence
  Ctsx.2
cctcttgatgttgattcggtctgc

SEQ ID NO: 9 - DNA, 20 bp, Artificial Sequence
  Ctsh.1
catggctgcaaaggaggtct

SEQ ID NO: 10 - DNA, 21 bp, Artificial Sequence
  Ctsh.2
ctgtcttcttccatgatgccc

(19) United States
(12) Patent Application Publication
(10) Pub. No.: US 2014/0186268 A1
(43) Pub. Date: Jul. 3, 2014

(54) Oxide Ferrimagnetics with Spinel Structure Nanoparticles and Iron Oxide Nanoparticles, Biocompatible Aqueous Colloidal Systems Comprising Nanoparticles, Ferriliposomes, and Uses Thereof

(71) Applicants: Jozef Stefan Institute, Ljubljana (SI); Institute of Strength Physics and Materials Science of Siberian Branch Russian Academy of Scie, Tomsk (RU)

(72) Inventors: Olga VASILJEVA, Domzale (SI); Volya I. ITIN, Tomsk (RU); Sergey G. PSAKHIE, Tomsk (RU); Georgy A. MIKHAYLOV, Ljubljana (SI); Mojca Urska MIKAC, Ljubljana (SI); Boris TURK, Skofljica (SI); Anna A. MAGAEVA, Tomsk (RU); Evgeniy P. NAIDEN, Tomsk (RU); Olga G. TEREKHOVA, Tomsk (RU)

(73) Assignees: Jozef Stefan Institute, Ljubljana (SI); Institute of Strength Physics and Materials Science of Siberian Branch Russian Academy of Scie, Tomsk (RU)

(21) Appl. No.: 14/172,401

(22) Filed: Feb. 4, 2014

Related U.S. Application Data
(63) Continuation of application No. PCT/RU2012/000632, filed on Aug. 3, 2012.

(30) Foreign Application Priority Data
Aug. 4, 2011 (RU) 2011132913
Aug. 4, 2011 (RU) PCT/RU2011/000574

(51) Int. Cl. A61K 49/08; A61K 49/00

(52) U.S. Cl. CPC A61K 49/08; A61K 49/0004
USPC 424/9.321; 424/9.32; 424/9.6

(57) ABSTRACT

The present invention relates to methods for producing oxide ferrimagnetics with spinel structure and iron oxide nanoparticles by soft mechanochemical synthesis using inorganic salt hydrates, oxide ferrimagnetics with spinel structure and iron oxide nanoparticles of ultra-small size and high specific surface area obtainable by the methods, biocompatible aqueous colloidal systems comprising oxide ferrimagnetics with spinel structure and iron oxide nanoparticles, carriers comprising oxide ferrimagnetics with spinel structure and iron oxide nanoparticles, and uses thereof in medicine.

### RELATED APPLICATIONS

This application is a Continuation application of International Patent Application PCT/RU2012/000632, filed on Aug. 3, 2012, which in turn claims priority to Russian Patent Applications No. RU 2011132913, filed Aug. 4, 2011 and International Patent Application PCT/RU2011/000574, filed Aug. 4, 2011, all of which are incorporated herein by reference in their entirety.

### FIELD OF THE INVENTION

The present invention relates to methods for producing oxide ferrimagnetics with spinel structure and iron oxide nanoparticles by soft mechanochemical synthesis using inorganic salt hydrates, oxide ferrimagnetics with spinel structure and iron oxide nanoparticles obtainable by the methods, stable and biocompatible aqueous colloidal systems comprising oxide ferrimagnetics with spinel structure and iron oxide nanoparticles, carriers comprising oxide ferrimagnetics with spinel structure and iron oxide nanoparticles, and uses thereof in medicine.

### BACKGROUND OF THE INVENTION

Magnetic resonance (MR) imaging (MRI) is a diagnostic method that enables tissue differentiation on the basis of different relaxation times. Contrast agents alter the relaxation times and are used to enhance the visualization of properties correlated with patient anatomy and physiology. The change in relaxation times depends on the contrast agent concentration as well as on magnetic field strength. Two types of contrast agents are known: T₁ contrast agents that shorten spin-lattice relaxation time of the nearby protons and T₂ contrast agents, which enhance spin-spin relaxation to darken the contrast media-containing structures. Contrast agent specificity is a desired property for enhancing signal-to-noise ratio at a site of interest and providing functional information through imaging. Natural biodistribution of contrast agents depends upon the size, charge, surface chemistry and administration route. Contrast agents may concentrate at healthy tissue or lesion sites and increase the contrast between the normal tissue and the lesion. In order to increase contrast, it is necessary to concentrate the agents at the site of interest and increase relativity. In addition, it is also desirable to increase the uptake of the agents by diseased cells in relation to healthy cells. Till now, superparamagnetic nanoparticles are used for MRI negative contrast, of which superparamagnetic iron oxide (SPIO) is the representative example.

WO 2008/127031 discloses magnetic resonance imaging contrast agents that comprise zinc-containing magnetic metal oxide nanoparticles. Optimized nanoparticles are proposed for conjugation with a bioactive material such as proteins, antibodies, and chemical materials. The proposed methods in WO 2008/127031 thus have limitations in accessibility of the materials used for such conjugation and careful analyses of their efficiency upon binding should be always performed.

The commercially available standard SPIOs, such as SHU 555A (Resovist®, Bayer HealthCare AG), are extremely strong enhancers of proton relaxation, but have very short useful half-lives after intravenous administration, as they are rapidly cleared from the blood within minutes and accumulate in the reticuloendothelial cells of the liver and spleen.

The properties and the further use of magnetic nanoparticles depends on the method by which they were obtained. The standard one step method of the preparation of magnetic nanoparticles by the method of co-precipitation was described before (Lopez et al., 2010; Morais et al., 2006). In contrast to that approach, the milling of reagents in the planetary mill will result in the ultrasmall sized nanoparticles with unique properties described in the current invention. Moreover, the use of saline crystal hydrates (proposed in the current invention) instead of the anhydrous salts that have been used in the work of Naiden et al., (Naiden et al., 2008), will change the solid-phase mechanism to a soft mechanochemical synthesis in aqueous media, resulting in a significantly increased reaction rate.

The main limiting factor for using of superparamagnetic nanoparticles in vivo is their low colloidal stability. To prevent nanoparticles aggregation, different methods by creating an electrostatic double layer have been developed. Mainly, those methods are based on the use of polymer surfactants functioning as a steric stabilizer, such as dimercaptosuccinic acid (DMSA) (Morais et al., 2004), polysaccharide polymer (dextran or dextran derivatives), starch, polyvinyl alcohol (PVA), polyethylene oxide (PEO), polyethylene glycol (PEG) or by modifying the isoelectric point with a citrate or silica coating (Bacri et al., 1990; Cornell, 1991). The most commonly used iron oxide nanoparticles are dextran coated, and are physiologically well tolerated (Babincova and Machova, 1999). There coating of magnetic nanoparticles by citric acid which form anionic monolayer on the particle surface was proposed by Morais et al., (Morais et al., 2006). However, the use of acid in state of buffers on the base of citrate salts could decrease the bioavailability of nanoparticles solution, particularly in the in vivo systems. Thus, to enable higher bioavailability of nanoparticles, a multiplex buffer containing HEPES, as a main component, and physiological pH (7.4) was developed in the current invention. Moreover, the salinity of this multiplex stabilizing buffer can be changed by variable concentration of saline component (NaCl). WO2009/002569 describes a procedure of effective polyurethane coupling of nanoparticles. Particles loaded and stable were obtained by this method. However, the described procedure is making impossible the coupling to the composite of additional components except nanoparticles. It can be critical point in use of such nanoparticles in more complicated systems, for example targeted delivery.

Another way is a coupling of nanoparticles by hydrophobic environment e.g polystyrene as described in WO2006/061835 or oleic acid (Lopez et al., 2010). Hydrophobic monolayer covered nanoparticles formed nanocrystals are stable and suspendable in non-polar and polar solvents. However, using nanoparticles thus stabilized in carrier systems, e.g. liposomes, there is possibility of incorporation of hydrophobic nanocrystals into the liposome bilayer which can destroy the liposome structure.

### SUMMARY OF THE INVENTION

Therefore, there is a need for novel paramagnetic nanoparticles, in particular iron oxide and ferrite nanoparticles having improved MR contrast properties and which offer the possibility of their targeted delivery, for use in the field of medicine, in particular diagnostics and treatment. There is also a need for stabilized formulations of iron oxide and ferrite nanoparticles as a prerequisite for use in medicine and/or for use as starting material for carriers comprising iron oxide nanoparticles, in particular ferriliposomes. In particular, there is a need for iron oxide nanoparticles with high colloidal stability in aqueous media as well as biocompatibility.

Compounds used for MRI contrast should be of a nanosize, stably dispersed both in aqueous media and in vivo environments and exhibit excellent MR contrast effects. Superparamagnetic nanoparticles currently suggested for the MRI have nanonsize and are stable for injection to the bloodstream what is very important to prevent thrombosis and blood vessels embolism. However, the use of such superparamagnetic nanoparticles for the high performance MRI applications is limited by their contrast properties.

### DETAILED DESCRIPTION OF THE PREFERRED EMBODIMENTS

In one embodiment, the present invention relates to a method for preparing oxide ferrimagnetics with spinel structures nanoparticles of ultra small size (below 30 nm) and high specific surface area (50-200 m²/g) according to formula (I):

MₓFe₃₋ₓO₄ (I)

wherein M is selected from Fe, Cu, Co, Ni, Mg and Mn, in particular M is Fe, and wherein 0≦x≦1, preferably 0.05≦x≦1,

comprising the following steps:
(i) mixing at least one inorganic salt of Fe, in particular FeCl₃, at least one inorganic salt of M, and an alkali hydroxide and/or alkali carbonate, in particular NaOH, and an inert diluent, in particular NaCl,
(ii) grinding and/or milling the mixture of (i),
(iii) optionally washing and/or drying the product of (ii), characterized in that the inorganic salts of Fe and M in step (i) are salt crystal hydrates.

In a preferred embodiment, a salt crystal hydrate selected from FeCl₃ 6H₂O; CoCl₂.6H₂O, CuCl₂.2H₂O is used etc.

The method of the invention surprisingly results in nanoparticles of ultrasmall size (less than about 50 nm, preferably less than about 30 nm) and a high specific surface area.

In a preferred embodiment, the oxide ferrimagnetics with spinel structure nanoparticles thus obtained are characterized by a diameter of less than about 50 nm, more preferably less than about 30 nm, even more preferably less than about 15 nm.

Such nanoparticles are particles of ultrasmall size.

In a preferred embodiment, the oxide ferrimagnetics with spinel structure nanoparticles thus obtained are characterized by specific surface area of about 50 to about 200 m²/g more preferably of about 100 to about 160 m²/g.

Such nanoparticles are characterized by a high specific surface area.

In a more preferred embodiment, the oxide ferrimagnetics with spinel structure nanoparticles thus obtained are characterized by

a) a diameter of less than about 50 nm, more preferably less than about 30 nm, even more preferably less than about 15 nm, and

b) specific surface area of about 50 to about 200 m²/g more preferably of about 100 to about 160 m²/g.

In a preferred embodiment, the diameter of the oxide ferrimagnetics with spinel structure nanoparticles thus obtained is at least about 1 nm, preferably at least about 3 nm.

In an especially preferred embodiment,

In a preferred embodiment, a salt crystal hydrate selected from FeCl₃.6H₂O; CoCl₂.6H₂O and CuCl₂.2H₂O is used.

In a further preferred embodiment, the crystal salt hydrate FeSO₄.7H₂O is used.

In a particularly preferred embodiment,

(i) the crystal salt hydrate FeSO₄.7H₂O, and

(ii) a crystal salt hydrate selected from FeCl₃.6H₂O; CoCl₂.6H₂O and CuCl₂.2H₂O is used.

In a preferred embodiment, grinding and/or milling is performed in a planetary mill.

In another preferred embodiment, the weight ratio of metal salt hydrates in total to the inert diluent, in particular NaCl, is about 1 to about 10 to about 1:1.5, preferably about 3:8.

In another preferred embodiment, the atmosphere in the planetary mill is vacuum, air or inert gas, in particular Ar.

In another preferred embodiment, the ratio of the mass of iron balls to the mass of reaction mixture according to step (i) is about 1:1 to about 50:1, preferably about 20:1.

In a preferred embodiment, the oxide ferrimagnetics with spinel structure are selected from the group consisting of Fe₃O₄, CoFe₂O₄, CuFe₂O₄, MnFe₂O₄, MgFe₂O₄, and NiFe₂O₄.

In another embodiment, the present invention relates to a method for preparing iron oxide nanoparticles according to formula (II):

Fe₃O₄ (II)

comprising the following steps:
(i) mixing FeCl₃.6H₂O and FeSO₄.7H₂O and NaOH, preferably in a ratio of about 2:1:8,
(ii) adding NaCl to the mixture of (i), preferably in a ratio of about 1:0.5 to about 1:4, preferably of about 1:2 of the mixture of (i) to NaCl,
(iii) grinding and/or milling the mixture of (ii),
   preferably grinding in a planetary mill, more preferably sealing the mixture of (ii) in steel cylinders with steel balls and grinding in a planetary mill milling, and
   wherein grinding is preferably performed for about 5 minutes to about 3 hours, in particular for about 30 minutes,
(iv) optionally washing and/or drying the product of (iii), characterized in that FeCl₃ and FeSO₄ in step (i) are in the form of salt crystal hydrates.

The reaction is as follows

FeSO₄+2FeCl₃+8NaOH→Fe₃O₄+Na₂SO₄+6NaCl+4H₂O

The method of the invention surprisingly results in nanoparticles of ultrasmall size (less than about 50 nm, preferably less than about 30 nm) and a high specific surface area. Moreover, as explained below, the method of the invention surprisingly results in novel iron oxide nanoparticles which are were proven to be ultrasmall and spherical with narrow size distribution and could successfully be formulated in a stable colloidal system. Moreover, the particles exhibit several fold higher relaxivities than commercial MRI contrast agents, resulting in ultra-sensitive MRI detection (FIG. 2a). Moreover, a 20-70% improvement in the r₂ relativity was found when compared to the best iron oxide-based nanoparticles described in the literature. Moreover, the nanoparticles are shown to be non-toxic (Examples 14 and 18), and are surprisingly effecting in targeted delivery in vivo.

In a preferred embodiment, the iron oxide nanoparticles thus obtained are characterized by a diameter of less than about 50 nm, more preferably less than about 30 nm, even more preferably less than about 15 nm

Such nanoparticles are particles of ultrasmall size.

In a preferred embodiment, the iron oxide nanoparticles thus obtained are characterized by specific surface area of about 50 to about 200 m²/g, more preferably of about 100 to about 160 m²/g.

Such nanoparticles are characterized by a high specific surface area.

In a more preferred embodiment, the iron oxide nanoparticles thus obtained are characterized by
a) a diameter of less than about 50 nm, more preferably less than about 30 nm, even more preferably less than about 15 nm, and
b) specific surface area of about 50 to about 200 m²/g more preferably of about 100 to about 160 m²/g.

In a preferred embodiment, the diameter of the iron oxide nanoparticles thus obtained is at least about 1 nm, preferably at least about 3 nm.

In one preferred embodiment, the salt crystal hydrate FeCl₃.6H₂O is used.

In a further preferred embodiment, the crystal salt hydrate FeSO₄.7H₂O is used.

In a particularly preferred embodiment, the crystal salt hydrates FeSO₄.7H₂O and

FeCl₃.6H₂O are used.

In another preferred embodiment, the ratio of the mass of iron balls to the mass of reaction mixture according to step (ii) is about 1:1 to about 50:1, preferably about 20:1.

In another preferred embodiment, the weight ratio of metal salt hydrates in total to the inert diluent, in particular NaCl, is about 1 to about 10 to about 1:1.5, preferably about 3:8.

The inert compound prevents heating of the reagent mixture.

In another preferred embodiment, the atmosphere in the planetary mill is vacuum, air or inert gas, in particular Ar.

In another preferred embodiment, mechanochemical synthesis in the planetary mill is performed in an MPV planetary mill.

In yet another preferred embodiment, mechanochemical synthesis in the planetary mill is performed in a planetary mill at about 30 g to about 100 g, preferably at about 60 g.

In another preferred embodiment, the washing of the nanoparticles according to the methods of the invention is performed by washing with distilled water.

In another preferred embodiment, the washing of the nanoparticles according to the methods of the invention is performed by washing on a filter.

In another preferred embodiment, the washing of the nanoparticles according to the methods of the invention is performed until the salts are completely removed from the filter.

In another embodiment, the present invention relates to an iron oxide nanoparticle or a oxide ferrimagnetics with spinel structure nanoparticle obtainable by any of the above methods of the invention.

In another embodiment, the present invention relates to an iron oxide nanoparticle or a oxide ferrimagnetics with spinel structure nanoparticle obtainable by any of the above methods of the invention.

In a preferred embodiment, the iron oxide nanoparticles and/or oxide ferrimagnetics with spinel structure nanoparticles obtainable by any of the above methods of the invention are characterized by a diameter of less than about 50 nm, more preferably less than about 30 nm, even more preferably less than about 15 nm.

Such nanoparticles are particles of ultrasmall size.

In a preferred embodiment, the diameter of the oxide ferrimagnetics with spinel structure nanoparticles and/or oxide ferrimagnetics with spinel structure nanoparticles obtainable by any of the above methods of the invention is at least about 1 nm, preferably at least about 3 nm.

In a preferred embodiment, the iron oxide nanoparticles and/or oxide ferrimagnetics with spinel structure nanoparticles obtainable by any of the above methods of the invention are characterized by specific surface area of about 50 to about 200 m²/g, more preferably of about 100 to about 160 m²/g.

Such nanoparticles are characterized by a high specific surface area.

In a more preferred embodiment, the iron oxide nanoparticles and/or oxide ferrimagnetics with spinel structure nanoparticles obtainable by any of the above methods of the invention are characterized by
a) a diameter of less than about 50 nm, more preferably less than about 30 nm, even more preferably less than about 15 nm, and
b) specific surface area of about 50 to about 200 m²/g, more preferably of about 100 to about 160 m²/g.

In another embodiment, the present invention relates to a method for preparing oxide ferrimagnetics with spinel structures nanoparticles according to formula (III):

CoₓFe₃₋ₓO₄ (III)

comprising the following steps:
mixing FeCl₃, CoCl₂, Na₂CO₃ and Ca(OH)₂, preferably in a ratio of about 2:1:3:1,
(ii) adding NaCl to the mixture of (i),
(iii) grinding and/or milling the mixture of (ii),
   preferably grinding in a planetary mill, more preferably sealing the mixture of (ii) in steel cylinders with steel balls and grinding in a planetary mill milling, and
   wherein grinding is preferably performed for about 5 minutes to about 3 hours, in particular for about 10 minutes to about 60 minutes,
(iv) optionally washing and/or drying the product of (iii),
   characterized in that FeCl₃, CoCl₂ in step (i) are in the form of salt crystal hydrates, and characterized adding NaCl to the mixture of (i) preferably in a ratio of about 1:0.5 to about 1:4, preferably of from about 1:2 to 1:3 of the mixture of (i) to NaCl.

The reaction is as follows:

2FeCl₃+CoCl₂+Ca(OH)₂+3Na₂CO₃=CoₓFe₃₋ₓO₄+CaCl₂+6NaCl+3CO₂↑+H₂O, wherein 0.1≦x≦0.99, preferably 0.6≦x≦0.98.

In one preferred embodiment, the salt crystal hydrate FeCl₃.6H₂O, and the salt crystal hydrate CoCl₂.6H₂O is used.

In another preferred embodiment, the ratio of the mass of iron balls to the mass of reaction mixture according to step (ii) is about 1:1 to about 50:1, preferably about 20:1.

The conditions for accomplishment of a given technical effect of the invention, which is a production of non-stoichiometric oxide ferrimagnetic nanoparticles with spinel structure are the strict adherence to the weight ratios of the mass of reaction mixture to the mass of inert component, preferably of about 1:2 to 1:3, the mass of powder to the mass of balls, preferably about 20:1, and the time for performing of mechanochemical synthesis of 10÷60 min.

In another preferred embodiment, the atmosphere in the planetary mill is vacuum, air or inert gas, in particular Ar.

In another preferred embodiment, mechanochemical synthesis in the planetary mill is performed in an MPV planetary mil.

1

In yet another preferred embodiment, mechanochemical synthesis in the planetary mill is performed in a planetary mill at about 30 g to about 100 g, preferably at about 60 g.

In another preferred embodiment, the washing of the nanoparticles according to the methods of the invention is performed by washing with distilled water.

In another preferred embodiment, the washing of the nanoparticles according to the methods of the invention is performed by washing on a filter.

In another preferred embodiment, the washing of the nanoparticles according to the methods of the invention is performed until the salts are completely removed from the filter.

In another embodiment, the present invention relates to an oxide ferrimagnetics with spinel structure nanoparticles obtainable by the above described

method by formula: CoₓFe₃₋ₓO₄, wherein 0.1≦x≦0.99, preferably 0.6≦x≦0.98.

In another embodiment, the present invention relates to an oxide ferrimagnetics with spinel structure nanoparticles obtainable by the above described method of the invention with the size of nanoparticles below 50 nm, in particular below 15 nm and high specific surface area in the range of 50-200 m²/g, in particular 100-160 m²/g.

In another embodiment, the present invention relates to a method for preparing oxide ferrimagnetic with spinel structure nanoparticles according to formula (IV):

MnₓFe₃₋ₓO₄ (IV)

comprising the following steps:
(i) mixing FeCl₃, MnO₂, and NaOH, preferably in a ratio of about 2:1:6,
(ii) adding NaCl to the mixture of (i),
(iii) grinding and/or milling the mixture of (ii),
   preferably grinding in a planetary mill, more preferably sealing the mixture of (ii) in steel cylinders with steel balls and grinding in a planetary mill milling, and
   wherein grinding is preferably performed for about 5 minutes to about 3 hours, in particular for about 30 minutes,
(iv) optionally washing and/or drying the product of (iii),
   characterized in that FeCl₃, in step (i) are in the form of salt crystal hydrates, and characterized adding NaCl to the mixture of (i), preferably in a ratio of about 1:1 to 1:3 of the mixture of (i) to NaCl.

The reaction is as follows:

2FeCl₃6H₂+MnO₂+6NaOH→MnₓFe₃₋ₓO₄+6NaCl+15H₂O+1/2O₂,

wherein 0.1≦x≦0.99, preferably 0.6≦x≦0.98.

In one preferred embodiment, only the salt crystal hydrate FeCl₃.6H₂O is used.

In another preferred embodiment, the ratio of the mass of iron balls to the mass of reaction mixture according to step (ii) is about 1:1 to about 50:1, preferably about 20:1.

In another preferred embodiment, the atmosphere in the planetary mill is vacuum, air or inert gas, in particular Ar.

In another preferred embodiment, mechanochemical synthesis in the planetary mill is performed in an MPV planetary mill.

In yet another preferred embodiment, mechanochemical synthesis in the planetary mill is performed in a planetary mill at about 30 g to about 100 g, preferably at about 60 g.

In another preferred embodiment, the washing of the nanoparticles according to the methods of the invention is performed by washing with distilled water.

In another preferred embodiment, the washing of the nanoparticles according to the methods of the invention is performed by washing on a filter.

In another preferred embodiment, the washing of the nanoparticles according to the methods of the invention is performed until the salts are completely removed from the filter.

In another embodiment, the present invention relates to an oxide ferrimagnetics with spinel structure nanoparticles obtainable by the above described method by formula: MnₓFe₃₋ₓO₄, wherein 0.1≦x≦0.99, preferably 0.6≦x≦0.98.

In another embodiment, the present invention relates to an oxide ferrimagnetics with spinel structure nanoparticles obtainable according the above described method of the invention to the methods of the invention with the size of nanoparticles below 50 nm, in particular below 15 nm and high specific surface area in the range of 50-200 m²/g, in particular 100-160 m²/g.

As shown in the Examples, the novel iron oxide nanoparticles of the invention were proven to be ultrasmall and spherical with narrow size distribution and high specific surface area and could successfully be formulated in a stable colloidal system. Moreover, the particles exhibit several fold higher relaxivities than commercial MRI contrast agents, resulting in ultra-sensitive MRI detection (FIG. 2a). Moreover, a 20-70% improvement in the r₂ relaxivity was found when compared to the best iron oxide-based nanoparticles described in the literature. Moreover, the nanoparticles are shown to be non-toxic (Examples 14 and 18), and are surprisingly effecting in targeted delivery in vivo.

As shown in the Example 2 the obtaining of the oxide ferrimagnetic nanoparticles with spinel structure which chemical composition is CoₓFe₃₋ₓO₄, where 0.1≦x≦0.99 provides the end product of high contrast properties at T₁ [symbol] T₂-relaxation time (FIGS. 26, 27).

In a preferred embodiment, the iron oxide nanoparticles or the oxide ferrimagnetics with spinel structure nanoparticles of the present invention have a diameter of about 1 to about 50 nm, in preferably of about 1 to about 30 nm, even more preferably of about 1 to 15 nm, in particular of about 3 to about 14 nm.

In another preferred embodiment, more than about 70% of the particles of the iron oxide nanoparticles or the oxide ferrimagnetics with spinel structure nanoparticles of the present invention have a diameter of less than about 8 nm. Preferably more than about 70%, more preferably more than about 80%, of the nanoparticles have a diameter of more than about 1 nm.

The novel iron oxide particles of the present invention were shown to exhibit such size distribution as shown in Example 1.

Thus, in another embodiment, the present invention relates to a population of iron oxide particles and/or oxide ferrimagnetics with spinel structure particles obtainable by above methods,

in particular wherein at least about 70%, more preferably at least about 80%, even more preferably at least about 90%, most preferably at least about 95% of the nanoparticles
(i) have a diameter of less than about 50 nm, more preferably less than about 30 nm, even more preferably less than about 15 nm, most preferably less than about 8 nm, and/or
(ii) have a diameter of at least about 1 nm, more preferably at least about 3 nm, even more preferably at least about 5 nm.

In another preferred embodiment, the negative surface zeta potential of the iron oxide nanoparticles or the oxide ferrimagnetics with spinel structure nanoparticles of the present invention is about 20 to about 30 mV, in particular about 28 mV, at pH 7.4 and 37° C.

The novel iron oxide particles of the present invention were shown to exhibit such zeta potential as shown in the Examples.

In another embodiment, the method for preparing iron oxide nanoparticles or oxide ferrimagnetics with spinel structure nanoparticles of the invention, further comprises the step of suspending the iron oxide nanoparticles or oxide ferrimagnetics with spinel structure nanoparticles in a biocompatible saline solution.

In a further embodiment, the present invention relates to a suspension of iron oxide nanoparticles of the present invention or oxide ferrimagnetics with spinel structure nanoparticles of the present invention, obtainable the method

In a further embodiment, the present invention relates to a method for preparing a biocompatible aqueous colloidal system comprising iron oxide nanoparticles or oxide ferrimagnetics with spinel structure nanoparticles comprising the following steps:
(i) suspending iron oxide nanoparticles or oxide ferrimagnetics with spinel structure nanoparticles, preferably powdered iron oxide nanoparticles or oxide ferrimagnetics with spinel structure nanoparticles, in a biocompatible saline solution,
(ii) disrupting the agglomerates formed in (i) by ultrasound, preferably disrupting with an ultrasonic disintegrator, in particular disrupting with an ultrasonic disintegrator at about 10 kHz to about 40 kHz, preferably at about 20 kHz,
(iii) separating the remaining agglomerates by centrifugation, preferably at about 100 to about 1000 g, more preferably at about 200 to about 800 g, even more preferably at about 500 g.

In a preferred embodiment of the present invention, the iron oxide nanoparticles or oxide ferrimagnetics with spinel structure nanoparticles of the present invention are used in step (i).

It was surprisingly shown, that stable colloidal systems could be generated, which do not result in agglomeration as a prerequisite for use in medicine. This could be shown in Example 3.

In another preferred embodiment, iron oxide nanoparticles may be used for in step (i) of the method. In particular, Feridex®, ferucarbotran, SHU 555C, or the zinc-containing particles disclosed in WO 2008/127031 may be used.

In another preferred embodiment of the present invention, the biocompatible saline solution comprises at least one buffering compound, in particular at least one buffering compound selected from citrate, HEPES ADA, Bicine, MES and Tris, in particular citrate and HEPES, in particular at a total concentration of buffering compounds of about 5 to about 100 mM, even more preferably of about 10 to about 70 mM.

In another preferred embodiment of the present invention, the biocompatible saline solution comprises about 50 to about 500 mM NaCl, preferably about 80 to about 400 mM NaCl, more preferably about 100 to about 350 mM NaCl, even more preferably about 108 mM NaCl.

In another preferred embodiment of the present invention, the biocompatible saline solution has a pH of about 4.0 to about 10.0, preferably has a pH of about 5.5 to about 9.0, more preferably has a pH of about 6.5 to about 8.5, even more preferably has a pH of about 7.4.

In another preferred embodiment of the present invention, the biocompatible saline solution comprises, in particular consists of 20 mM sodium citrate, 108 mM NaCl, and 10 mM HEPES, and wherein the pH of the biocompatible saline solution is about 7.4, which is equal to physiological pH.

In another preferred embodiment of the present invention, the biocompatible saline solution is sterile.

In another embodiment of the present invention, the invention relates to a biocompatible aqueous colloidal system comprising iron oxide nanoparticles or oxide ferrimagnetics with spinel structure nanoparticles obtainable by the methods of the invention for preparing a biocompatible aqueous colloidal system.

In a further embodiment, the present invention relates to a method for preparing ferriliposomes comprising:
(i) hydrating a dry lipid film comprising at least one lipid, preferably at least one phospholipid with a biocompatible aqueous colloidal system of the invention or a suspension of the invention,
(ii) generating liposomes, preferably by extrusion or sonification, more preferably by extrusion, and
(iii) optionally washing and/or isolating the ferriliposomes.

In a preferred embodiment, step (iii) comprises:
(a) separating non-encapsulated iron-oxide particles, preferably by gel filtration,
(b) isolating the ferriliposomes magnetically, in particular using a magnetic separator, and
(c) optionally washing the ferriliposomes and/or suspending the ferriliposomes in a solution, in a particular a biocompatible saline solution.

In a further embodiment, the present invention relates to a ferriliposome
(i) obtainable by a method for preparing ferriliposomes according to the present invention and/or
(ii) comprising
   (a) at least one iron oxide nanoparticle and/or oxide ferrimagnetics with spinel structure nanoparticle of the present invention, or at least one iron oxide nanoparticle and/or oxide ferrimagnetics with spinel structure nanoparticle of the suspension of the present invention, or at least one iron oxide nanoparticle and/or oxide ferrimagnetics with spinel structure nanoparticle of the biocompatible aqueous colloidal system of the present invention, and
   (b) at least one lipid, in particular at least one phospholipid.

The ferriliposomes of the present invention are surprisingly useful for targeted delivery in vivo and in vitro as shown in Examples 11, 13, 18, 19 and 24 as well as sections “Ferriliposome delivered JPM-565 inhibits growth of mammary tumour lesions” and “Efficacy of ferriliposomes as an MRI-visible drug delivery system in vivo” in the Examples. Moreover, the ferriliposomes are shown to be non-toxic (Example 17).

In a preferred embodiment, the method for preparing ferriliposomes of the invention, or the ferriliposome of the invention are characterized by:
(a) that the at least one phospholipid is a phosphatidylcholine, in particular L-a-phosphatidylcholine, and/or
(b) that the ferriliposomes of the invention, or the dry lipid film used in the method for preparing ferriliposomes of the invention further comprise a PEGylated lipid, in particular 1,2-distearoyl-sn-glycero-3-phosphoethanolamine-N-[methoxy(polyethylene glycol)-2000].

As could be shown in FIG. 7, macrophage uptake of the PEGylated ferriliposomes of the present invention is greatly reduced. Modification of the liposome surface with PEG is known to greatly reduce the opsonization of liposomes and their subsequent clearance by the reticuloendothelial (mononuclear-phagocyte) system, resulting in a substantially prolonged circulation half-life. This was confirmed in the cellular experiment underlying FIG. 7. Because of their size, hydrophobic and hydrophilic character, biocompatibility, together with the internal hollow space (see FIG. 7), the system of ferriliposomes of the present invention enables simultaneous encapsulation of iron oxide nanoparticles or oxide ferrimagnetics with spinel structure nanoparticles with other substances, such as therapeutically active agents, and their subsequent targeted delivery in the organism, in particular mammals, more preferably humans.

As shown in the Examples, the ferriliposomes according to the present invention are magnet-sensitive and can target both the tumour and its microenvironment. The nanosized ferriliposomes display outstanding magnetic resonance T₂ contrast properties (r₂=573-1286 s⁻¹ mM⁻¹), both in vitro and in vivo in transgenic tumour bearing mice, enabling drug delivery to be monitored noninvasively. Successful tumour microenvironment targeting and uptake of a probe administered by ferriliposomes were visualized in vivo. Targeted delivery of an inhibitor of tumour promoting proteases to the mouse mammary tumour and its microenvironment substantially reduced tumour size compared to systemic delivery of the same drug.

In a preferred embodiment, the ferriliposome of the present invention further comprises at least one therapeutically active agent and/or at least one diagnostically active agent and/or at least one agent allowing targeting of the ferriliposome, preferably
(1) wherein the therapeutically active agent and/or diagnostically active agent is encapsulated in the liposome or is integrated in the bilayer, and/or
(ii) wherein the at least one therapeutically active agent is selected from:
   a toxin,
   a chemotherapeutic agent,
   in particular an alkylating agent or/and an anti-metabolite or/and a plant alkaloid or/and a taxane or/and a topoisomerase inhibitor or/and a antineoplastic agent, more preferably doxorubicin,
   a radioactive agent,
   a protease inhibitor, in particular a cathepsin inhibitor, more preferably JPM-565,
   an apoptosis-inducing agent, and
   an anti-inflammatory agent,
      in particular a non-steroidal anti-inflammatory agents,
         preferably selected from a salicylate, propionic acid derivative, acetic acid derivative, enolic acid derivative, and fenamic acid derivative, a selective COX-2 inhibitor, and a sulphonanilide, or
      in particular a steroidal anti-inflammatory agents, preferably a glucocorticoid,
   and/or
(iii) wherein the at least one diagnostically active agent is selected from:
   a radioactive agent,
   a paramagnetic agent,
   a PET-imagable agent,
   an MRI-imagable agent,
   a fluorophore, in particular Alexa Fluor,
   a chromophore,
   a phosphorescing agent,
   a chemoluminescent agent, and
   a bioluminescent agent.

As shown in the Examples, doxorubicin and a Cathepsin-inhibitor could successfully be targeted to the peri-tumoral region of mouse breast cancer, resulting in significant tumour growth reduction without any adverse effect (see Examples, section “Ferriliposome delivered JPM-565 inhibits growth of mammary tumour lesions”, Examples 9 and 15).

In one embodiment, the present invention relates to a ferriliposome of the present invention, or an iron oxide nanoparticle of the present invention or a oxide ferrimagnetics with spinel structure nanoparticle of the present invention, for use in medicine, in particular
(i) for use in the diagnosis of diseases, preferably neoplastic, neuronal and/or inflammatory diseases, and/or
(ii) for use as MRI contrast agents.

In another embodiment, the present invention relates to a ferriliposome of the present invention, or an iron oxide nanoparticle of the present invention or an oxide ferrimagnetics with spinel structure nanoparticle of the present invention, for the preparation of a medicament and/or diagnostic, in particular
(i) for the diagnosis of diseases, preferably neoplastic, neuronal and/or inflammatory diseases, and/or
(ii) for the preparation of a MRI contrast agent.

In another embodiment, the present invention relates to a method of diagnosing and/or treating a disease, a preferably a neoplastic, a neuronal and/or an inflammatory disease, comprising administering to a patient in need thereof a diagnostically an/or therapeutically effective amount of a ferriliposome of the present invention, or an iron oxide nanoparticle of the present invention or an oxide ferrimagnetics with spinel structure nanoparticle of the present invention. In a preferred embodiment, the patient is a mammal, in particular a human.

In a preferred embodiment, the method further comprises applying a magnetic field by a magnet, in particular applying the magnetic field locally at the site to be diagnosed and/or treated. Typically the magnetic field is in the range of about 0.3 to about 4.5 Tesla, in particular in the range of about 1.0 to about 3.5 Tesla.

The present invention further provides iron oxide and oxide ferrimagnetics with spinel structure nanoparticles as well as ferriliposomes which allow targeted delivery of the nanoparticles and ferriliposomes respectively, to a site of interest using a magnetic field. Thereby, the iron oxide and oxide ferrimagnetics with spinel structure nanoparticles as well as ferriliposomes a magnetic resonance imaging represent (MRI)-visible drug delivery systems. The iron oxide and oxide ferrimagnetics with spinel structure nanoparticles as well as ferriliposomes are useful for determining the distribution of drugs using MRI, as well as organ-, tissue-, and/or site-specific drug delivery.

The oxide ferrimagnetics with spinel structure nanoparticles and iron oxide nanoparticles may be formulated and administered as a pharmaceutical composition.

The present invention therefore also relates to a pharmaceutical composition comprising at least one ferriliposome of the present invention, or iron oxide nanoparticle of the present invention or an oxide ferrimagnetic with spinel structure nanoparticle of the present invention or biocompatible aqueous colloidal system of the present invention.

The pharmaceutical composition of the present invention comprises therapeutically and/or diagnostically effective amounts oxide ferrimagnetics with spinel structure nanoparticles and/or iron oxide nanoparticles and/or ferriliposomes of the invention and generally an acceptable pharmaceutical carrier, diluent or excipient, e.g. sterile water, physiological saline, bacteriostatic saline, i.e. saline containing about 0.9% mg/ml benzyl alcohol, phosphate-buffered saline, Hank's solution, Ringer's-lactate, lactose, dextrose, sucrose, trehalose, sorbitol, mannitol, and the like. In a preferred embodiment, a formulation in a biocompatible saline solution according to the present invention, optionally comprising further excipients and additives, may be used. In another preferred embodiment, a biocompatible aqueous colloidal system according to the present invention, optionally comprising further excipients and additives may be used. The composition is generally a colloid, dispersion or suspension. It can be administered systemically, intravenously, orally, subcutaneously, intramuscularly, pulmonary, by inhalation and/or through sustained release administrations. Preferably, the composition is administered systemically, in particular intravenously.

The term “therapeutically effective amount” generally means the quantity of a therapeutically active agent, where applicable, which results in the desired therapeutic effect. A typical dosage range is from about 0.01 μg to about 1000 mg per application.

The term “diagnostically effective amount” generally means the quantity of a oxide ferrimagnetics with spinel structure nanoparticle and/or iron oxide nanoparticle and/or ferriliposome of the invention which results in the desired diagnostic effect without causing unacceptable side-effects. A typical dosage range is from about 0.01 μg to about 1000 mg per application.

Generally, the administration of the oxide ferrimagnetics with spinel structure nanoparticle and/or iron oxide nanoparticle and/or ferriliposome and/or pharmaceutical composition to a patient is one made one or several times, for example one or several times per day, or one or several times a week, or even during longer time periods as the case may be. For diagnostic purposes, a single administration may be sufficient.

In a further embodiment, the present invention relates to a carrier comprising at least one iron oxide nanoparticle and/or oxide ferrimagnetics with spinel structure nanoparticle of the present invention, or at least one iron oxide nanoparticle and/or oxide ferrimagnetics with spinel structure nanoparticle of the suspension of the present invention, or at least one iron oxide nanoparticle and/or oxide ferrimagnetics with spinel structure nanoparticle of the biocompatible aqueous colloidal system of the present invention, preferably wherein the carrier is selected from a nanotube, a liposome, a lipoplex, a polymersome, a micell, a nanogel, a mesoporous silica nanoparticle, a dendrimer, and a nanoshell, in particular the carrier is a liposome.

In a further embodiment, the present invention relates to a kit comprising:
(a) at least one ferriliposome of the present invention, and/or at least one iron oxide nanoparticle and/or at least one oxide ferrimagnetics with spinel structure nanoparticle of the present invention, and
(b) at least one magnet.

In one embodiment, the iron oxide nanoparticles can be used as T₂ MR contrast agents and/or negative MR contrast agents. The negative T₂ effect is very strong, as the MR signal diminishes in the vicinity of the presence of the contrast agent on T₂-weighted MR images.

In one embodiment, the oxide nanoparticles facilitate an increased contrast in the MRI images and/or an increased signal. In a preferred embodiment, the increased signal can be translated to shorter acquisition times, and/or higher spatial resolution and/or a reduction in dose of the contrast agent.

In another embodiment, the oxide ferrimagnetics nanoparticles with spinel structure according to the formula CoₓFe₃₋ₓO₄ can be used as T₁ and T₂ contrast agents.

“About” according to the present invention is understood as meaning the experimental error range, in particular ±5% or ±10%.

“Nanoparticle” according to the present invention is understood as particle with a diameter in at least one dimension exceeding at least about 1 nm, preferably at least about 10 nm, more preferably at least about 20 nm. In a preferred embodiment, a nanoparticle has a diameter in at least two dimensions, preferably in three dimensions exceeding at least about 1 nm, preferably at least about 10 nm, more preferably at least about 20 nm, as determined by dynamic light scattering, as for example described in the Examples. In a preferred embodiment, a nanoparticle is spherical. In a further preferred embodiment, a nanoparticle is less than about 1 μm, preferably less than 150 nm in diameter in at least one dimension, preferably in at least two dimensions, preferably in three dimensions.

“Iron oxide nanoparticle” according to the present invention is understood as nanoparticles consisting of at least about 80%, preferably at least about 90%, more preferably at least about 95%, even more preferably at least about 99% iron oxide. In particular, iron oxide is understood as Fe₃O₄ or FeO.Fe₂O₃.

A “spinel” according to the present invention is understood as compounds of the formula [A_{y}B_{2-y}]O₄, wherein A is a divalent metal, preferably Fe²⁺, Cu, Co, Ni, Mg or Mn, in particular A is Fe²⁺, and wherein B is a 3- or 4-valent metal, for example Al, Fe³⁺, V, Cr, Ti, in particular B is Fe³⁺, and wherein y is 0 or 1.

“Oxide ferrimagnetics with spinel structure” according to the present invention are understood as spinel of the formula M²⁺Fe³⁺₂O₄ or MIIO.Fe₂O₃, wherein M is a divalent metal, preferably Fe²⁺, Cu, Co, Ni or Mn, in particular Fe²⁺. Suitable oxide ferrimagnetics with spinel structures comprise for example Fe₃O₄, CuFe₂O₄, NiFe₂O₄, MgFe₂O₄ and CoFe₂O₄, preferably Fe₃O₄ and nonstoichiometric oxide ferrimagnetics with spinel structure comprise for example CoₓFe₃₋ₓO₄ and MnₓFe₃₋ₓO₄, wherein 0.1≦x≦0.99, preferably 0.6≦x≦0.98.

A carrier according to the present invention is understood as a chemical entity with a length in at least one dimension exceeding at least about 1 nm, preferably at least about 10 nm, more preferably at least about 100 nm, and which allows covalent or non-covalent binding of further moieties. In a preferred embodiment of the present invention, the carrier is selected from a nanotube, a liposome, a lipoplex, a polymersome, a micell, a nanogel, a mesoporous silica nanoparticle, a dendrimer, and a nanoshell, in particular the carrier is a liposome. In the case of a liposome, the nanoparticles and, where applicable, a further therapeutic and/or diagnostic moiety may be encapsulated within the liposome, or, where applicable, the further therapeutic and/or diagnostic moiety may integrated in the bilayer of the liposome. The generation of liposomes may be performed by methods known to a skilled person. In particular, the liposomes may be generated by extrusion as described in Example 4, or by sonification. In other embodiment, like e.g. dendrimers, the nanoparticles of the present invention, and, where applicable, the therapeutic and/or diagnostic moiety are bound covalently to the dendrimer, preferably via a linker. The synthesis and use of a nanotube, a liposome, a lipoplex, a polymersome, a micell, a nanogel, a mesoporous silica nanoparticle, a dendrimer, and a nanoshell are for example described in (Orive et al., 2009) A “liposome” according to the present invention is understood as vesicle made of a lipid bilayer. A liposome may comprise one or more lipids, in particular phospholipids, more preferably phosphatidylcholine. A liposome may comprise further lipids, preferably PEGylated lipids, even more preferably. In a preferred embodiment, the lipid may be present. Such lipids are described in Example 4. A liposome may be unilamellar or multilamellar. In case of multilamellar liposomes, it may comprise 2, 3, 4, 5 or more lamelles.

“Ferriliposome” according to the present invention is understood as liposome comprising one or more iron oxide nanoparticles and/or oxide ferrimagnetics with spinel structure nanoparticles of the invention. In a preferred embodiment, the nanoparticle(s) are located in the hollow space of the liposome. A ferriliposome may comprise further diagnostically and/or therapeutically active agents as described above. Also, a ferriliposome may be coated with an optionally functionalized polymer. For example, a ferriliposome may be coated with dextran or functionalized dextran. Ferriliposomes coated with Alexa Fluor-conjugated dextran are described in Example 4. The ferriliposomes typically have a diameter of about 20 nm to about 1 μm, preferably of about 50 nm to about 500 nm, more preferably of about 80 nm to about 200 nm, in particular preferably of about 90 nm to about 110 nm. A ferriliposome may be unilamellar or multilamellar. In case of multilamellar ferriliposomes, it may comprise 2, 3, 4, 5 or more lamelles.

A “therapeutic moiety” according to the present invention is a chemical moiety, which is capable of exhibiting a therapeutic effect when administered to a patient in need thereof in an effective amount. The therapeutic moiety is preferably selected from:

a chemotherapeutic agent,
in particular an alkylating agent or/and an anti-metabolite or/and a plant alkaloid or/and a taxane or/and a topoisomerase inhibitor or/and a antineoplastic agent, more preferably doxorubicin,

a radioactive agent,

a protease inhibitor, in particular a cathepsin inhibitor, more preferably JPM-565,

an apoptosis-inducing agent, and

an anti-inflammatory agent,
in particular a non-steroidal anti-inflammatory agents,
   preferably selected from a salicylate, propionic acid
   derivative, acetic acid derivative, enolic acid derivative, and
   fenamic acid derivative, a selective COX-2 inhibitor, and a
   sulphonanilide, or
in particular a steroidal anti-inflammatory agents,
   preferably a glucocorticoid.

It is understood, that the selection of the therapeutic moiety depends on the disease to be treated.

A “diagnostic moiety” according to the present invention is a chemical moiety, which can be detected. Preferably, the chemical moiety can be detected in vitro, ex vivo, or in vivo, preferably in vivo. The diagnostic moiety is preferably selected from:

a radioactive agent,

a paramagnetic agent,

a PET-imagable agent,

an MRI-imagable agent,

a fluorophore, in particular Alexa Fluor,

a chromophore,

a phosphorescing agent,

a chemiluminescent agent, and

a bioluminescent agent.

“Biocompatible” according to the present invention is understood as a solution that will not induce any undesirable local or systemic response in the animal, in particular human, to which it is administered. In a preferred embodiment, the administration is systemic, in particular intravenous.

### BRIEF DESCRIPTION OF THE DRAWINGS

FIG. 1: Characterization of the iron oxide particles and ferriliposomes.

FIG. 1A: Transmission electron micrographs (TEM, EM-125) of iron oxide nanoparticles; The inset shows the corresponding electron diffraction pattern. FIG. 1B: Size distribution of iron oxide nanoparticles and their average size (D=6.65 nm); FIG. 1C: Field emission gun scanning electron microscopy of the aqueous colloidal system of iron oxide nanoparticles; FIG. 1D: Dynamic light scattering measurement of iron oxide colloidal dispersion showing the distribution of diameter of nanoparticle clusters and their average size (D=38.95 nm); FIG. 1E: Ferriliposomes and atomic force microscopy image of ferriliposomes;

FIG. 1F: Ferriliposome size determination by dynamic light scattering and their average size (D=92.3 nm);

FIG. 2: MR contrast properties of electrostatically stabilized iron oxide nanoparticles:

FIG. 2A: Spin-lattice 1/T₁ and spin-spin 1/T₂ relaxation rates of 39 nm and 57 nm iron oxide nanoparticles versus different commercially available MR contrast agents (Feridex® (AMAG Pharmaceuticals), Magnevist® (Bayer HealthCare AG)). Symbols are measured values, and lines are fits to the equation: 1/Tᵢ=rᵢ·c+1/Tᵢ₀, where rᵢ is the relaxivity, c the concentration, Tᵢ₀ is the relaxation rate of 1% agarose and i is 1 for T₁ and 2 for T₂. Relaxivity rates r₁ and r₂ were obtained by comparison of the measured and theoretical values; FIG. 2B: MR images of agarose phantoms at different concentrations of 39 nm and 57 nm iron oxide nanoparticles: T₁-weighted (TE=8.5 ms, TR=400 ms) and T₂-weighted MR images (TE=60 ms, TR=2000 ms). Based on these results iron oxide nanoparticles could be used as positive (T₁) and negative (T₂) targeted MRI contrast agents; FIG. 2C: T₂-weighted MR image (TE=60 ms, TR=2000 ms) of four phantom-probes containing 1% agarose (1 and 3), 3.4 mM iron oxide nanoparticles injected in the centre of the 1% agarose gel probe (2), and 3.4 mM iron oxide nanoparticles diffused into 1% agarose under an applied magnetic field (4) together with signal intensity profiles along lines i and ii. A small probe is a phantom containing solution of CuSO4.H2O (5);

FIG. 3: In vivo detection of ferriliposomes targeting and release:

FIG. 3A: T₂-weighted MR images (TE=60 ms, TR=2000 ms, slice thickness=1 mm) of an MMTV-PyMT transgenic mouse in vivo before, 1 hour and 48 hours after intraperitoneal administration of 200 μl ferriliposome solution (concentration 3.4 mM) followed by 1 hour of magnetic field application on the lower right mammary gland. The tumour tissue possessing high MR signal appeared yellow red on original T₂-weighted images. The homogeneous darkening on the tumour exposed to the 0.33 T magnet (white arrow) indicates preferential accumulation of ferriliposomes. The insert shows the region of MR imaging denoted by a red rectangle; FIG. 3B: Fluorescence images of primary MMTV-PyMT tumour cells and MEFs incubated with Alexa Fluor 555™-functionalized ferriliposomes for 3 hours at 37° C. The scale bar corresponds to 20 μM. Data are representative of three separate experiments; FIG. 3C: Targeted delivery of ferriliposomes carrying D-luciferin into double transgenic mice expressing luciferase (FVB.luctg/+;PyMTtg/+) produced a high-intensity luciferase signal associated with the tumour. D-luciferin loaded ferriliposomes were intraperitoneally administrated into FVB.luctg/+;PyMTtg/+ mice with (targeted FL) and without (non targeted FL) magnet application. 24 hours after injection the magnet was detached and mice were imaged with an IVIS® Imaging System (5 minutes, IVIS® 100 Series). Luciferase activity was specifically detected only in the tumour region exposed to the 0.33 T magnet (black arrow), indicating an efficient D-luciferin release only from the targeted ferriliposomes in vivo. The scale is in photons/sec/sm²/sr;

FIG. 4: The anti-tumour effect of ferriliposome targeted cysteine proteases inhibitor JPM-565:

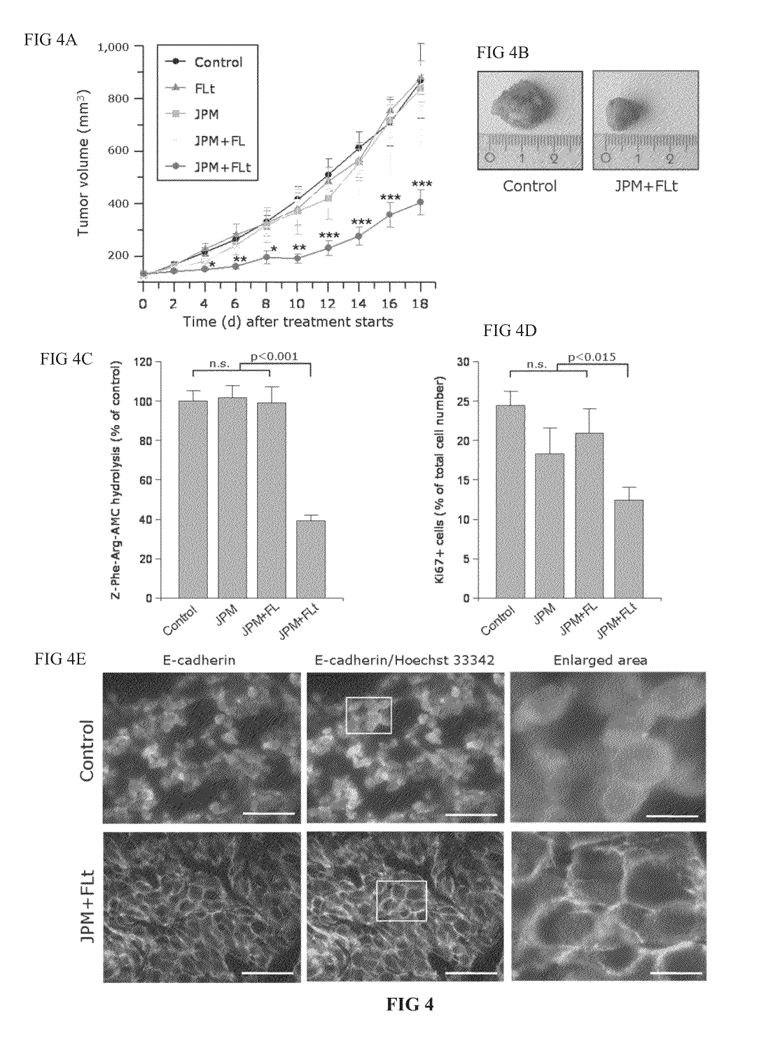

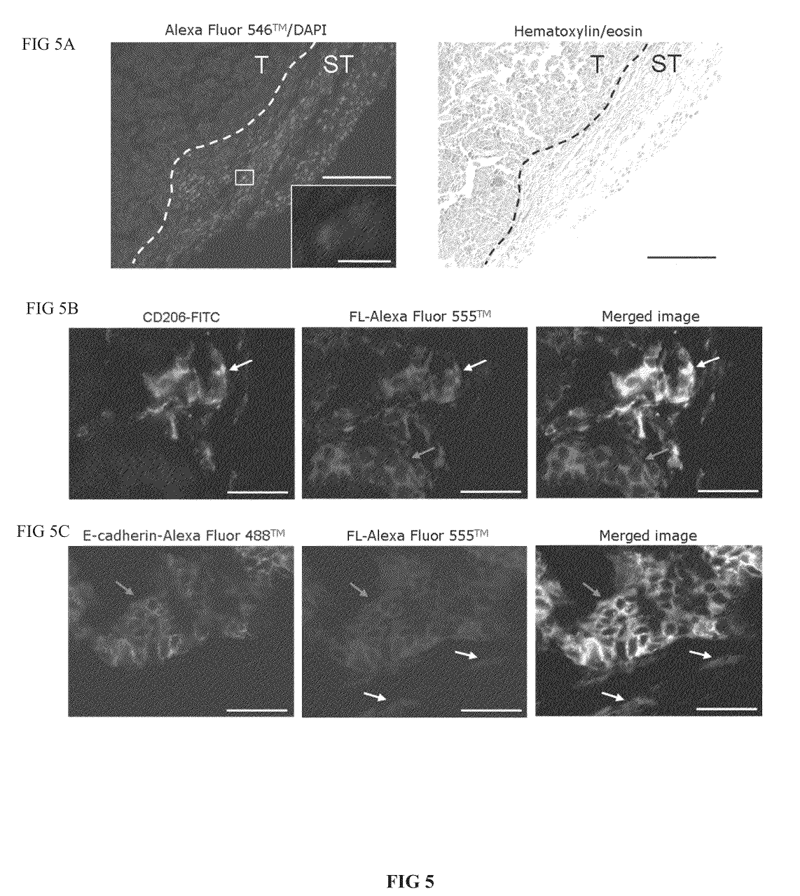

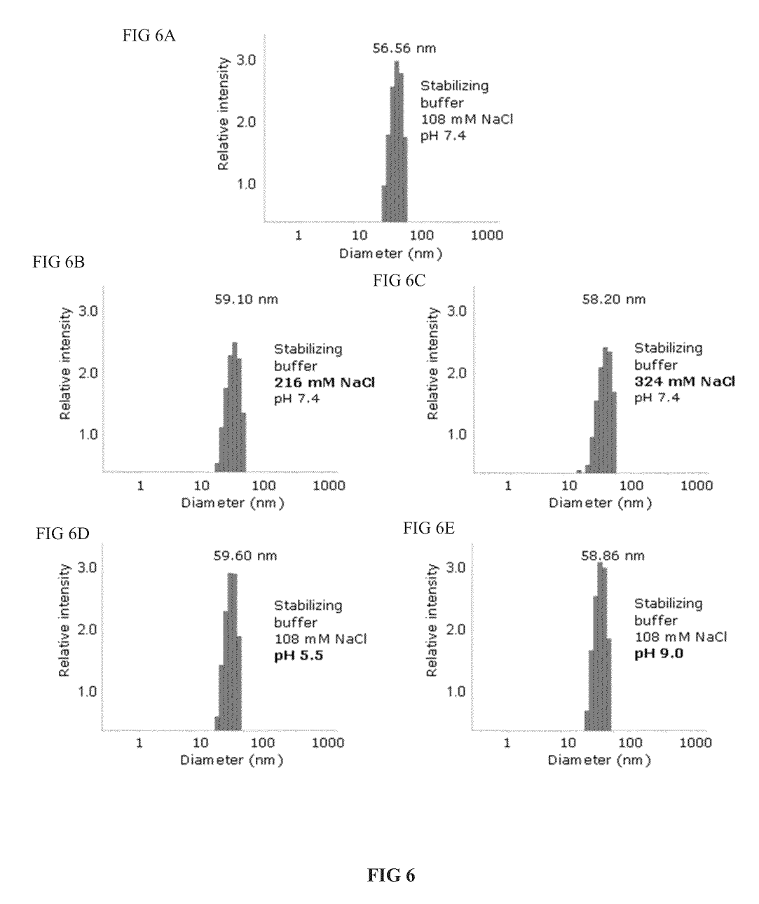

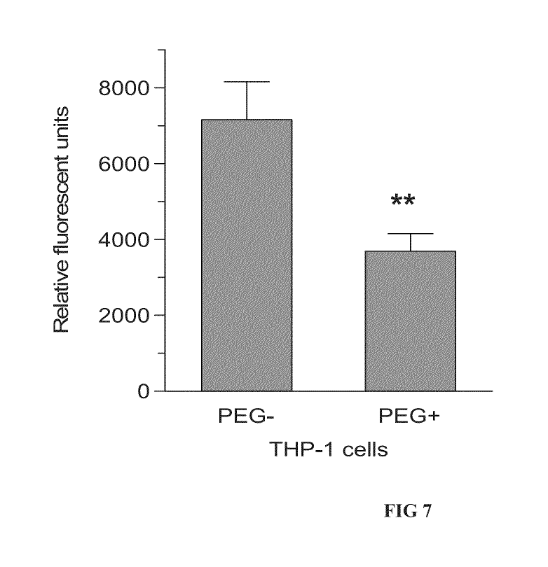

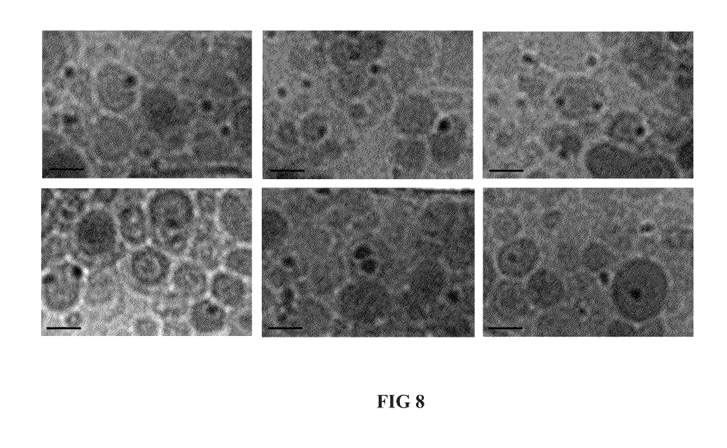

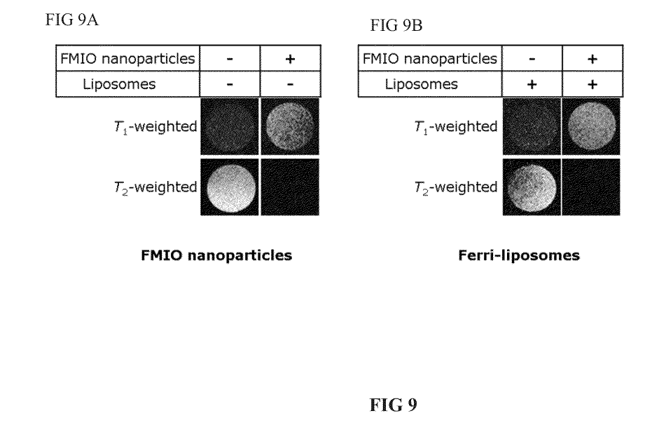

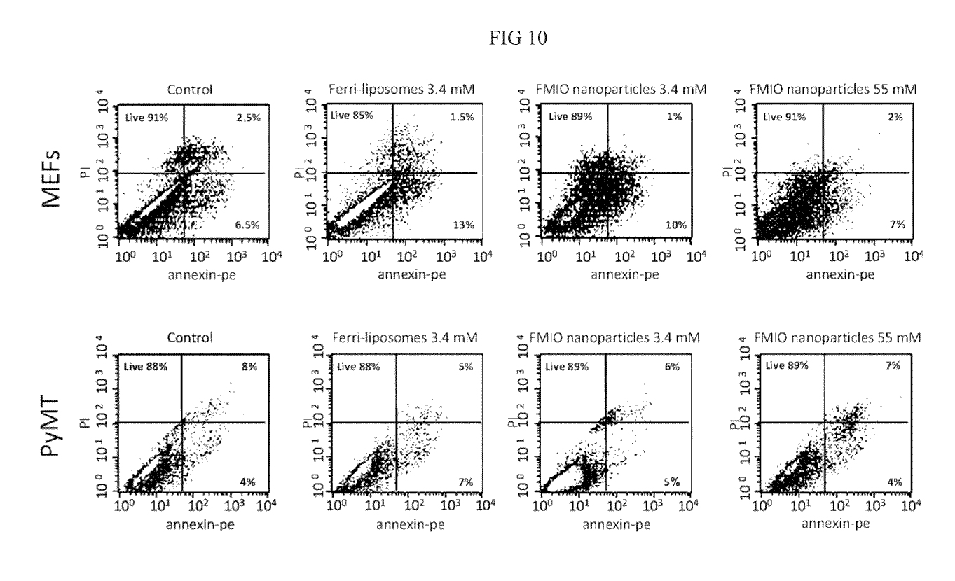

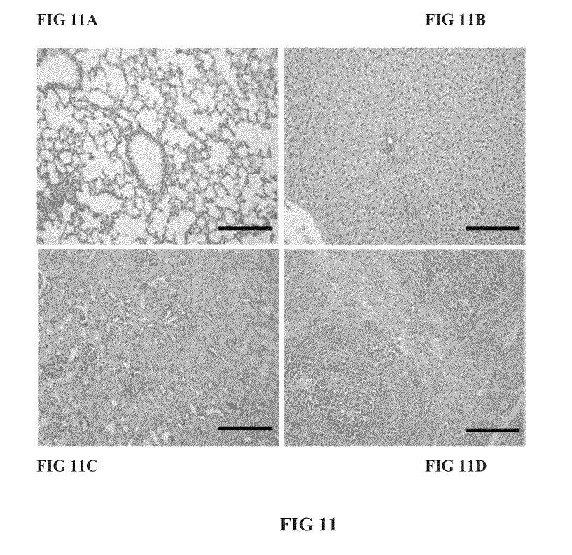

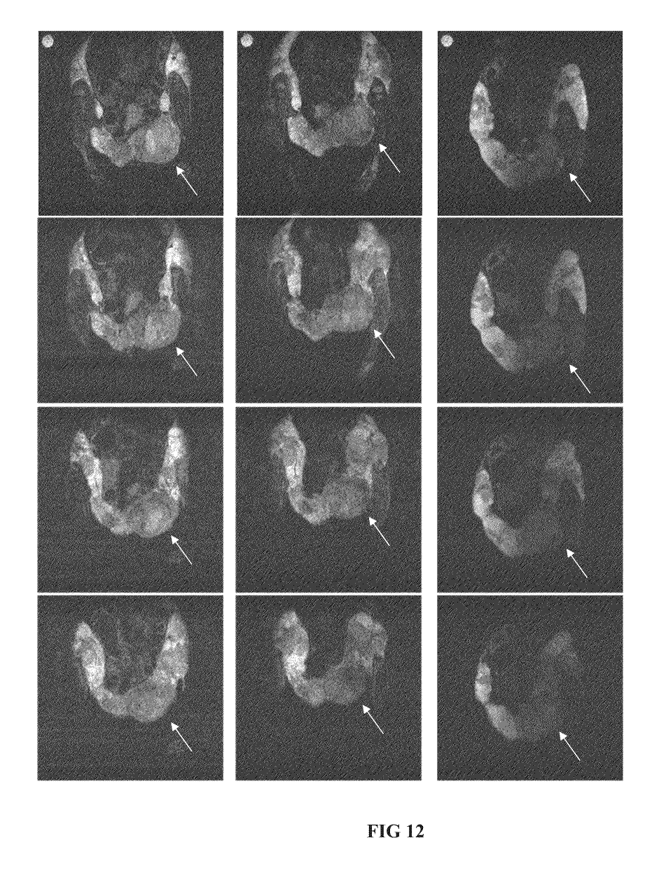

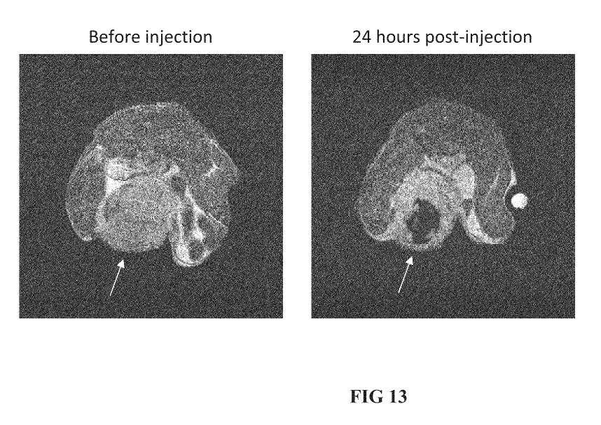

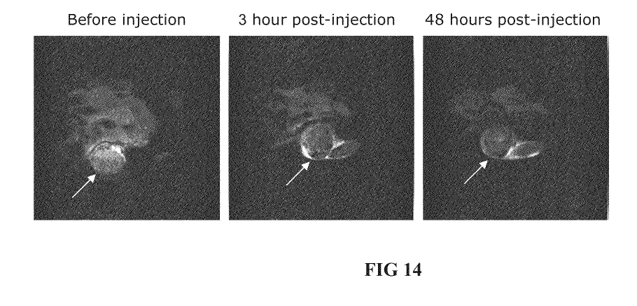

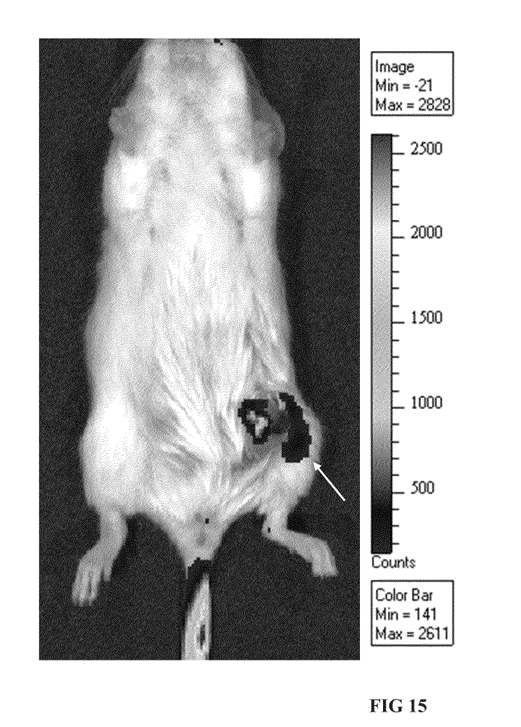

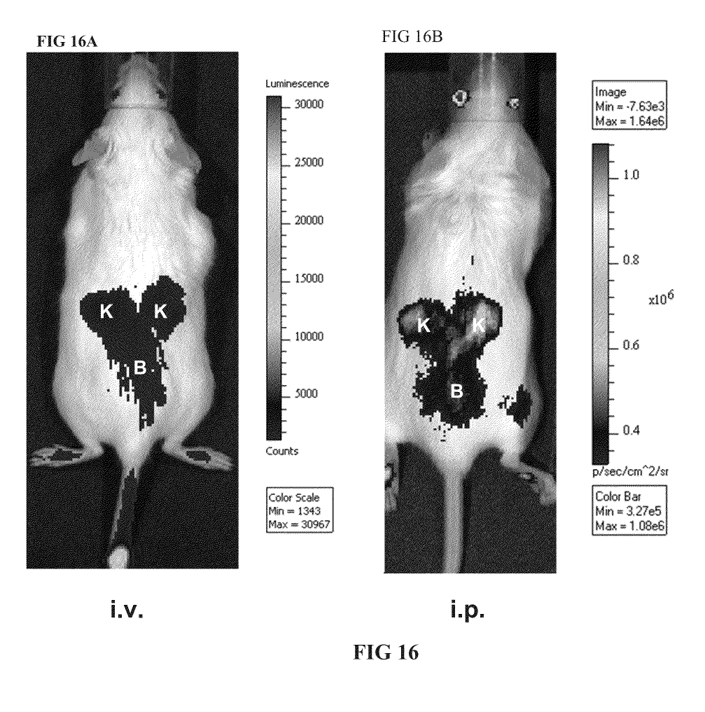

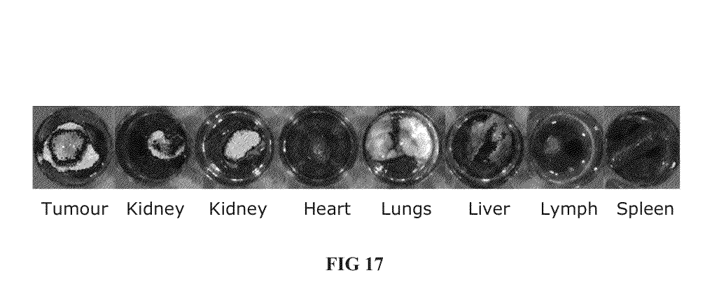

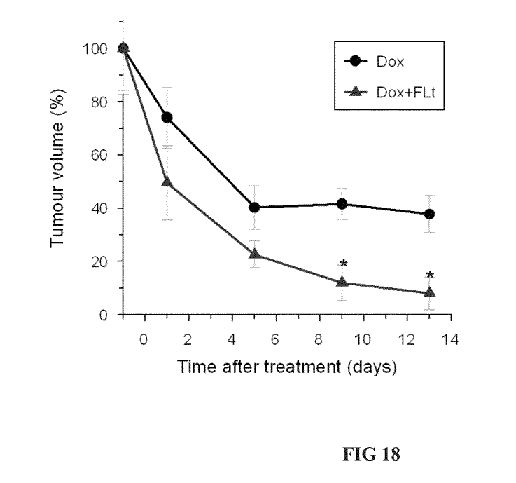

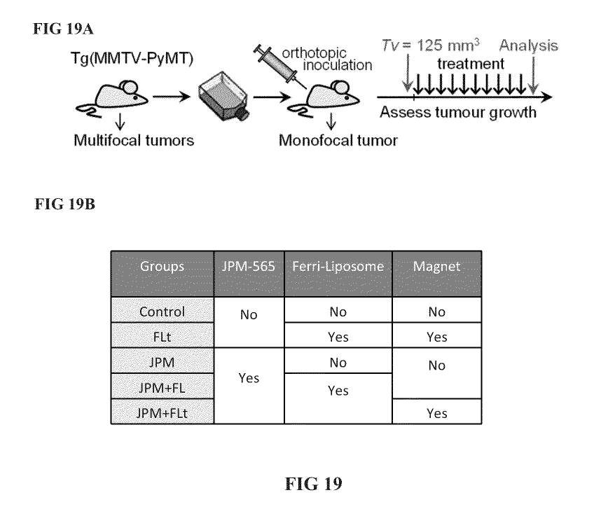

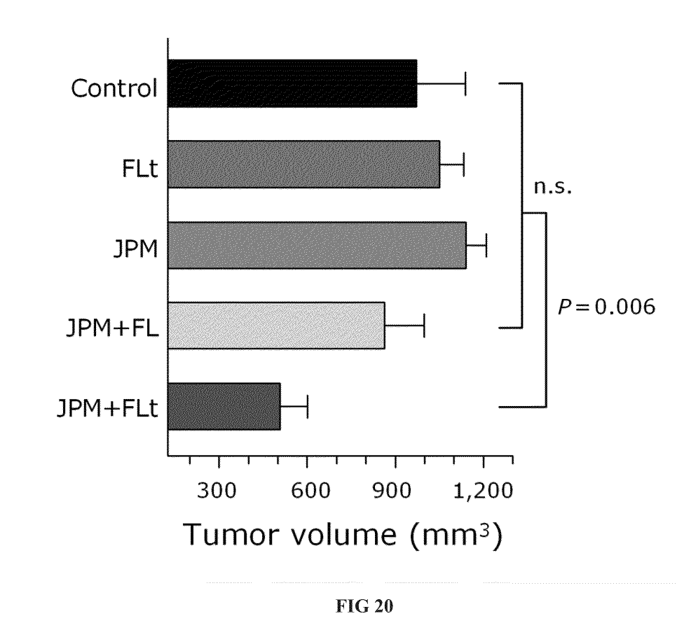

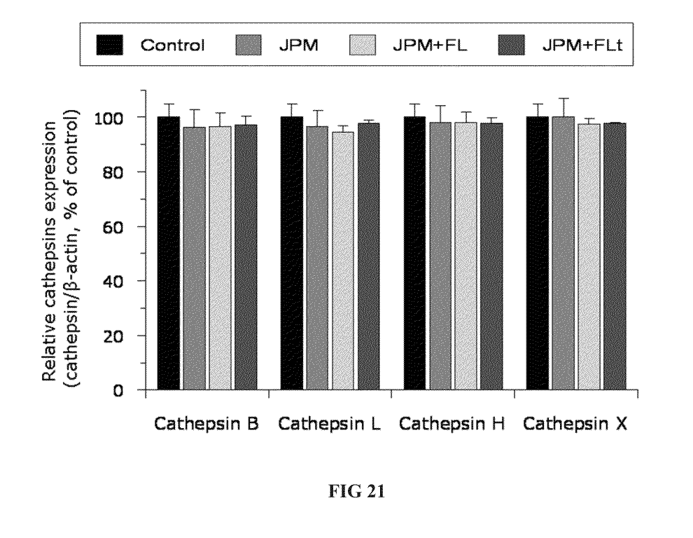

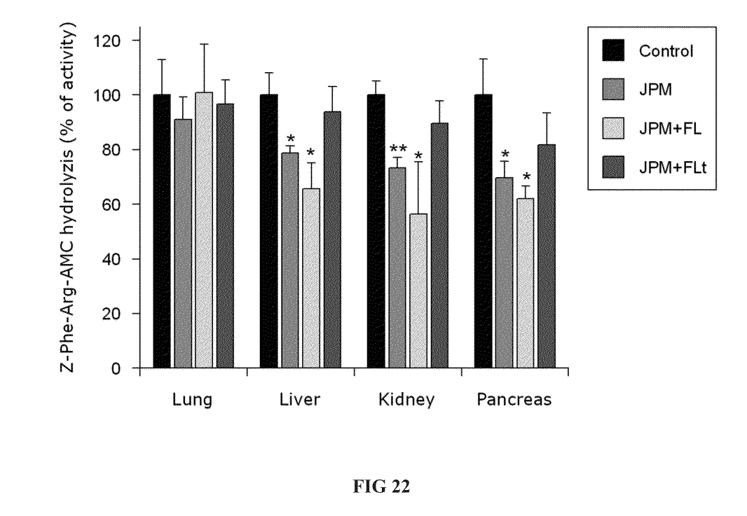

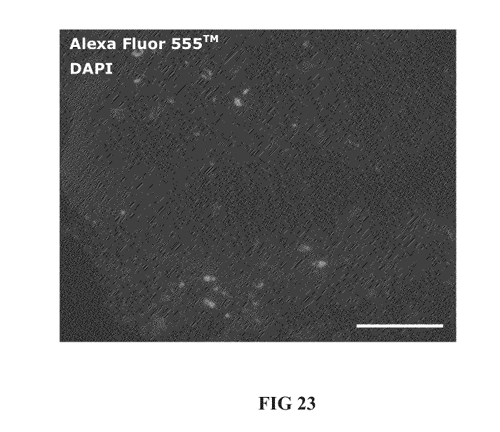

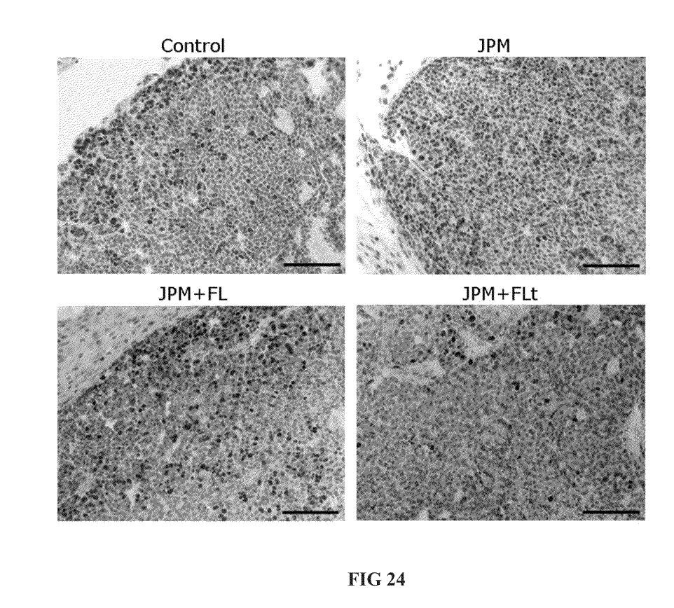

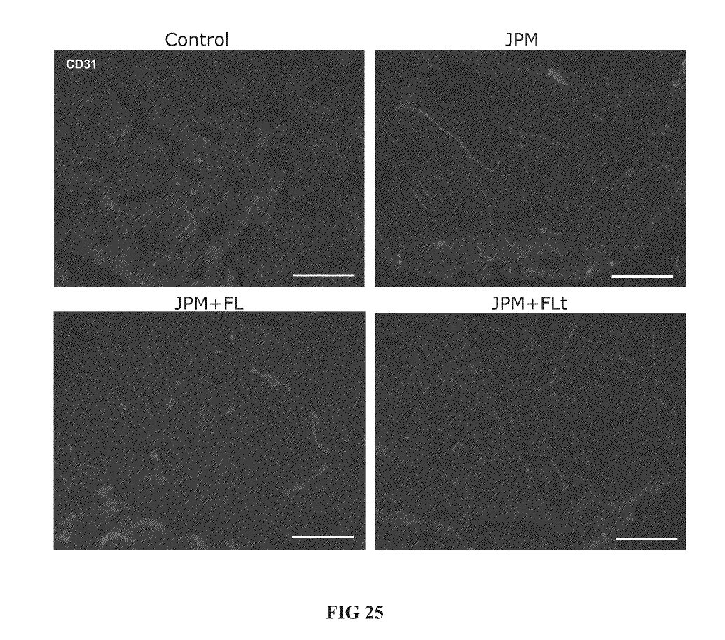

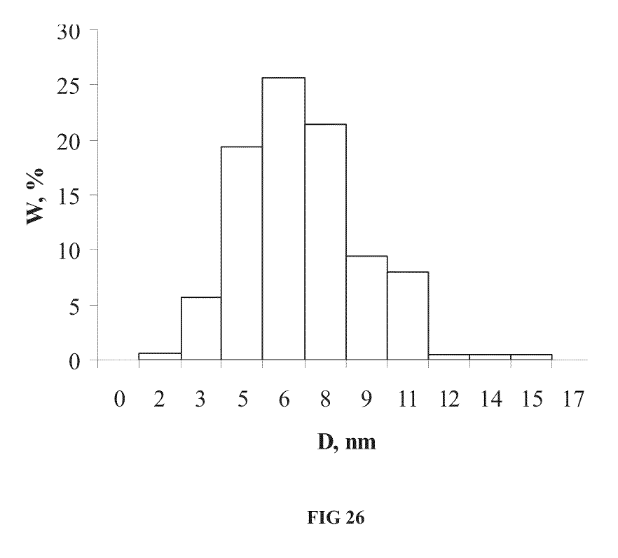

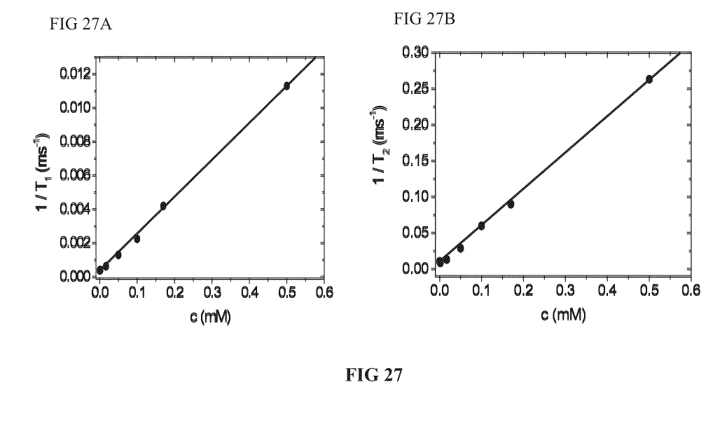

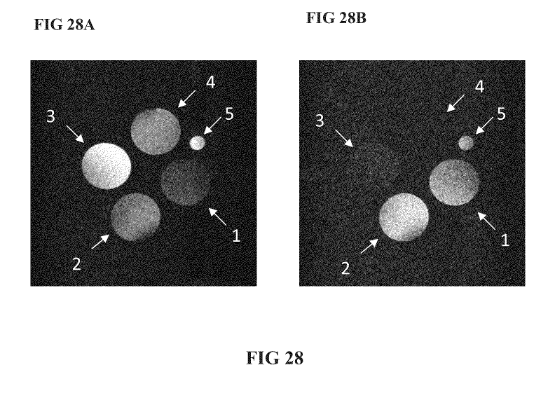

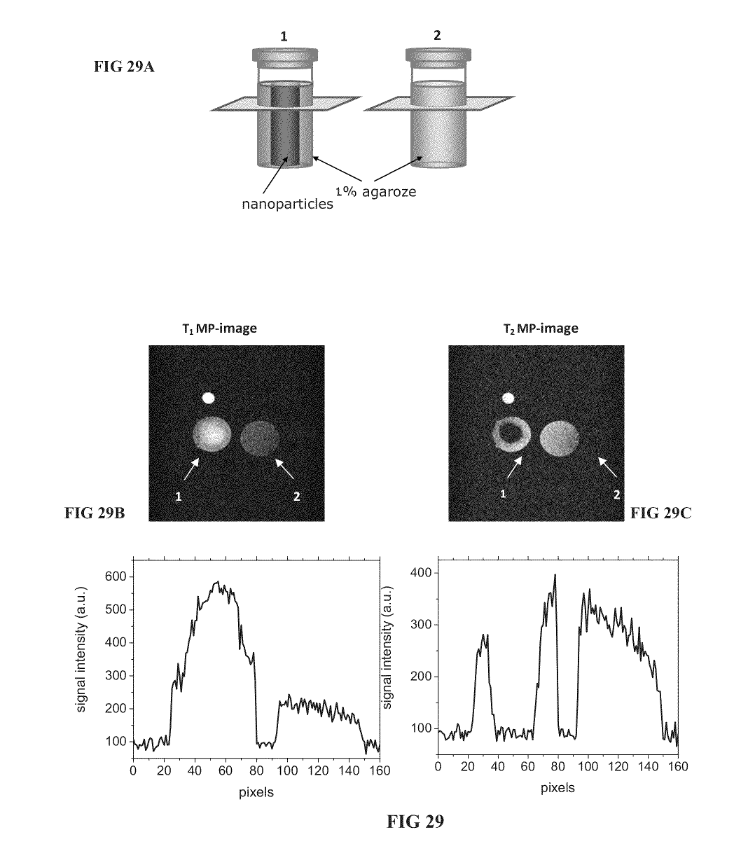

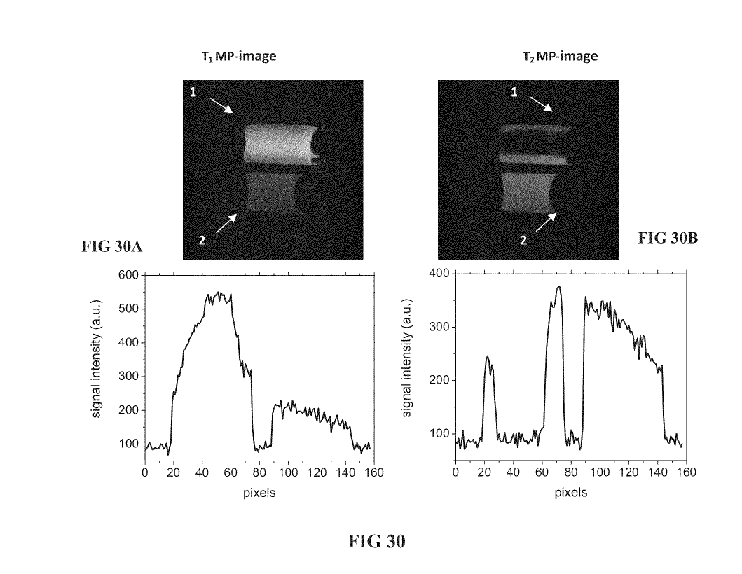

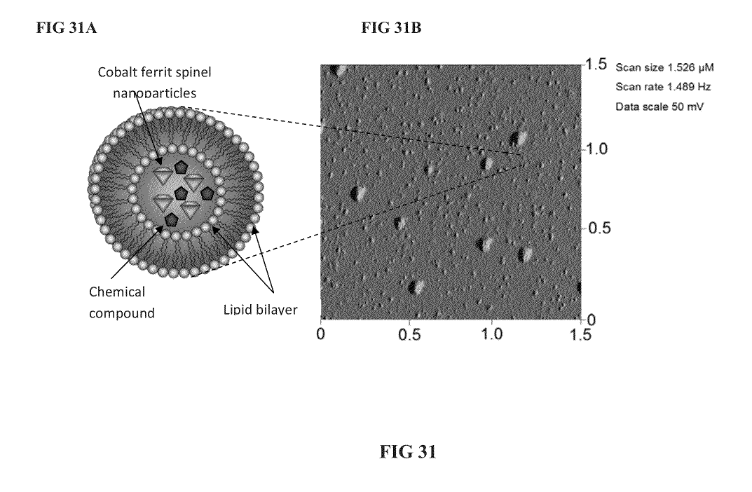

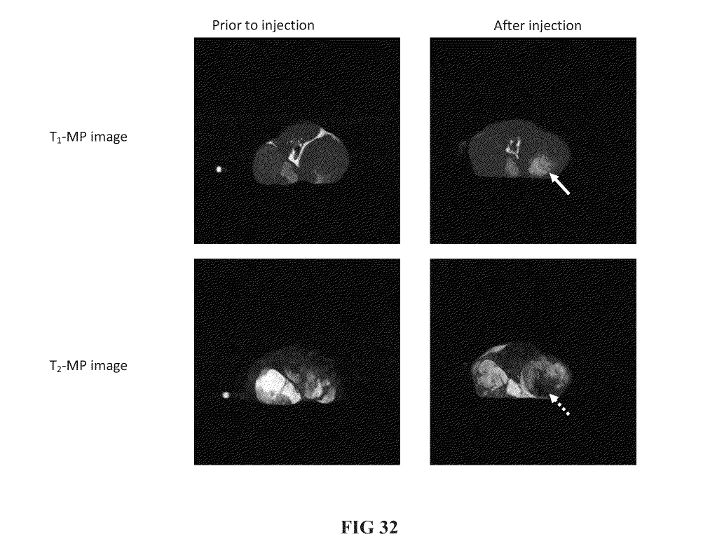

FIG. 4A: Tumour volumes for each treatment day. *P<0.05, **P<0.01 and **P<0.001, compared with the other groups; FIG. 4B:
Representative images of the single tumours excised from the mice of control and treated by ferriliposome delivered JPM-565 groups; FIG. 4C: Activity of cysteine cathepsins in tumour tissue after JPM-565 administration. Tumours were prepared h after the final injection, and cysteine cathepsin activity was measured by hydrolysis of the fluorogenic substrate Z-Phe-Arg-AMC; FIG. 4D-FIG. 4E: 24 hours after the last treatment, the degree of proliferation and E-cadherin expression were compared by immunostaining of Ki-67 and E-cadherin staining; FIG. 4D: Quantification of Ki67+ cells as percentage of total cells. The percentage of Ki67+ cells was calculated from 10 high-power fields (HPF) per animal by computer-assisted data analyses using the histoquest software (TissueGnostics). Data are presented as means and standard errors, n=5. Statistics were analyzed using Student's t-test; FIG. 4E: E-cadherin-stained images (green fluorescence) of control tumours and tumours treated by JPM-565 targeted by ferriliposomes are shown in the left panels, with E cadherin/E-cadherin/Hoechst 33342 (green/blue) merged images in the middle panels. A higher-magnification image outlined by the white rectangle illustrates the different patterns of E-cadherin localization—cytosolic in control tumours and cell membrane associated in “JPM+FLt” treated tumours. The scale bar corresponds to 100 μm and 25 μm in the higher magnification images;

FIG. 5: In vivo detection of fluorescent ferriliposomes in tumours:

FIG. 5A: In vivo detection of tumour-targeted ferriliposomes administered intraperitoneally. Left panel: fluorescent tissue images confirm the presence of Alexa Fluor 546™-functionalized ferriliposomes (red fluorescence) in the tumour microenvironment stained with DAPI (nuclear stain, blue fluorescence). Inserted is a higher-magnification image of an individual cell of the tumour stroma from the region outlined by the white rectangle. Haematoxylin and eosin (H&E) staining of the corresponding section is shown in the right panel. Stromal (ST) and tumour (T) compartments of tumour tissue are indicated and their boundary is demarcated by a dotted line; FIG. 5B:, Co-staining of tumour-associated macrophages (CD206 marker for alternatively activated macrophages; green fluorescence) and with Alexa Fluor 555™ labeled ferriliposomes (red fluorescence) after double intravenous injection of ferriliposomes; FIG. 5C: Co-staining of tumour cells (epithelial marker E-cadherin; green fluorescence) with Alexa Fluor 555™ labeled ferriliposomes (red fluorescence). The images demonstrate the cellular uptake of Alexa Fluor 555™ functionalized ferriliposomes both by stroma (white arrows) and tumour cells (magenta arrows). The scale bar corresponds to 200 μm in a, 40 μm in b, c and 20 μm in the insert;

FIG. 6: Colloidal stability of the iron oxide nanoparticles under various pH and salt concentrations:
An iron oxide nanoparticle suspension was prepared in a stabilizing buffer (FIG. 6A) and its colloidal stability was tested at different ionic strengths: 216 mM NaCl (FIG. 6B), 324 mM NaCl (FIG. 6C); and pH values: pH 5.5 (FIG. 6D), pH 9.0 (FIG. 6E). Average sizes of stabilized iron oxide nanoparticles were measured by dynamic light scattering (DLS);

FIG. 7: Effect of liposome PEGylation on macrophage uptake:
Fluorescence intensity was taken as the measure of uptake of non-PEGylated (PEG−) and PEGylated (PEG+) liposomes loaded with Alexa Fluor 565™ by THP-1 cells differentiated into the macrophages (** p<0.01);

FIG. 8: Transmission electron microscope (TEM) images of ferriliposomes; the sample of ferriliposomes prepared in vitreous ice suspended over a holey carbon substrate; the TEM images (NanoImaging services, Inc., CA, USA) ferriliposomes; the scale bar is 100 nm;

FIG. 9: Comparison of MRI contrast properties of non-encapsulated iron oxide nanoparticles and ferriliposomes comprising such nanoparticles:
MR images of non-encapsulated iron oxide nanoparticles (FIG. 9A) and ferriliposomes (FIG. 9B) as compared to the stabilizing buffer or empty liposomes solution, respectively: T₁-weighted (TE=8.5 ms, TR=400 ms) and T₂-weighted MR images (TE=60 ms, TR=2000 ms), at 0.085 mM concentration of iron oxide nanoparticles. Based on these results both iron oxide nanoparticles and ferriliposomes could be used as positive (T₁) and negative (T₂) targeted MRI contrast agents;

FIG. 10: Analysis of cellular toxicity of ferriliposomes and iron oxide nanoparticles:
Mouse embryonic fibroblasts (MEFs) and primary PyMT tumour cells were incubated with ferriliposomes containing 3.4 mM nanoparticles, or 3.4 mM and 55 mM iron oxide nanoparticles at 37° C. for 24 hours. Cells were then labeled with Annexin V-PE in the presence of 5 μg/ml of propidium iodide. Fluorescence intensity was measured by flow cytometry and data were analyzed by the Cell Quest software. No significant difference in cell death between different groups was detected. Results are means of 3 independent experiments, expressed as percentage of the total cell number;

FIG. 11: Histo-pathological analysis of the organs of healthy animals after infusion of iron oxide nanoparticles:
A single dose of iron oxide nanoparticles (500 mg/kg) was administered to normal healthy rats in a similar manner as in all other experiments. At day 7 after the administration histo-pathological analysis of FIG. 11A, lung, FIG. 11B, liver, FIG. 11C, kidney and FIG. 11D, spleen was performed by hematoxylin and eosin. No morphological changes were observed in treated animals. The scale bar corresponds to 100 μm;

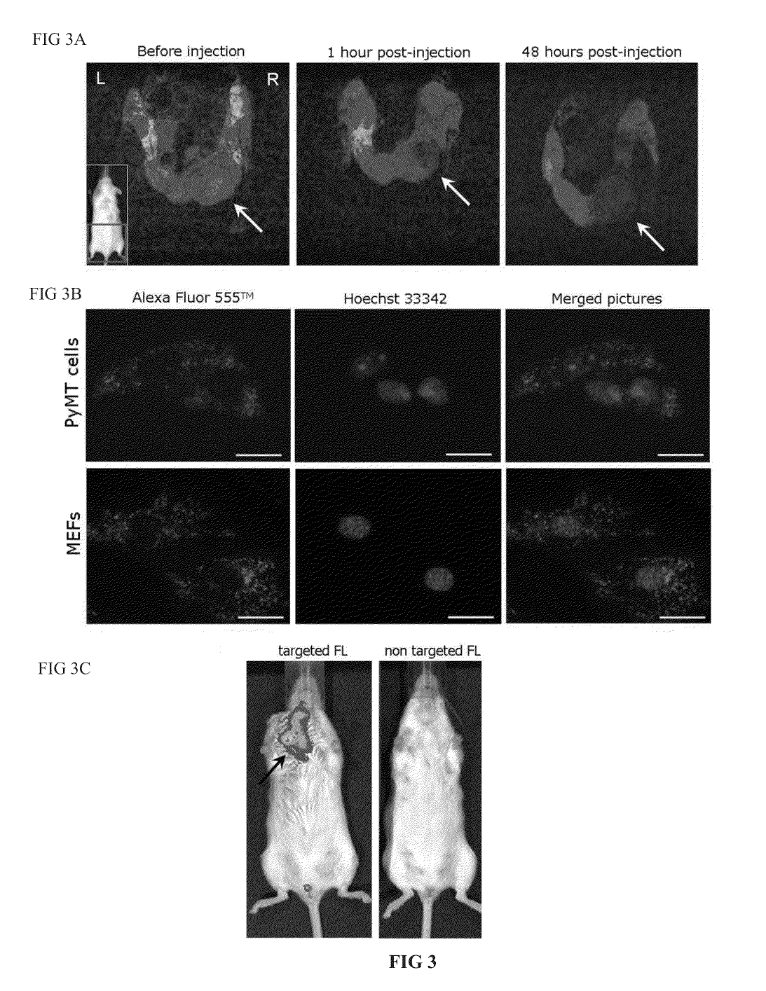

FIG. 12: In vivo MR detection of tumor targeted ferriliposomes introduced by intraperitoneal injection:
Four consecutive coronal slices of T₂-weighted MR images (TE=60 ms, TR=2000 ms, slice thickness=1 mm) of an MMTV-PyMT transgenic mouse in vivo before, 1 hour and 48 hours after intraperitoneal administration of 200 μl ferriliposome solution (concentration 3.4 mM) followed by 1 hour of magnetic field (0.33 T) application to the lower right mammary gland (white arrows). A clear heterogeneous darkening could be qualitatively observed on the tumour exposed to the magnet, indicating preferential accumulation of ferriliposomes. The phantom that was used as a reference is shown in the left upper corners of the first slices. The bottom line slices are the gray scale images of the FIG. 3A;

FIG. 13: In vivo MR detection of tumor targeted ferriliposomes introduced by intraperitoneal injection;
T₂-weighted MR images of transversal slices (TE=60 ms, TR=2000 ms, slice thickness=1 mm) of transplanted mammary tumor in vivo: before and 24 hours post-injection of 200 μl ferriliposome solution followed by magnetic field (0.33 T) application. A clear darkening was qualitatively observed on the tumour exposed to the magnet for 24 hours (white arrows), indicating preferential accumulation of ferriliposomes in the center of the tumor;

FIG. 14: In vivo MRI monitoring of ferriliposomes distribution after intravenous ferriliposomes administration:
T₂-weighted MR images (TE=60 ms, TR=2000 ms, slice thickness=1 mm) of an MMTV-PyMT transgenic mouse in vivo before, 3 hours and 48 hours after intravenous administration of 200 μl ferriliposome solution (concentration 3.4 mM) followed by 1 hour of magnetic field application on the lower left mammary gland. The tumour tissue exhibiting high MR signal appears yellow-red on T₂-weighted images. The homogeneous darkening on the tumour exposed to the 0.33 T magnet (white arrow) indicates preferential accumulation of ferriliposomes;

FIG. 15: Targeted delivery of ferriliposomes carrying D-luciferin into transgenic mice expressing luciferase (FVB.luctg/+):
D-luciferin loaded ferriliposomes were administered intravenously into the FVB.luctg/+ mice followed by magnetic field application. 24 hours after injection the magnet was detached and mice were imaged with an IVIS® Imaging System (5 minutes, IVIS® 100 Series). Luciferase activity was specifically detected only in the tumour region exposed to the 0.33 T magnet (black arrow), indicating an efficient D-luciferin targeting and release in vivo. The scale is in photons/sec/sm²/sr;

FIG. 16: Elimination of ferriliposomes in vivo:
Luminescent signal was measured in dorsal scans of luciferase transgenic mice 24 h after intravenous (FIG. 16A, i.v.) or intraperitoneal (FIG. 16B, i.p.) injection of D-luciferin loaded ferriliposomes and magnetic targeting with an IVIS® Imaging System. Bioluminescence was detected in both cases from the urinary tract of mice, indicating that nanoparticles were eliminated by renal clearance very similarly regardless of the ferriliposome administration route;

FIG. 17: Ex vivo luminescence imaging of ferriliposomes bio-distribution after dissection:
D-luciferin loaded ferriliposomes were administered intravenously into the FVB.luctg/+ mouse followed by magnetic field application. 24 hours after injection the magnet was detached and mouse was sacrificed and the organs harvested and imaged with an IVIS® Imaging System (2 minutes, IVIS® 100 Series). A significantly higher luciferase activity was detected in the tumour and kidneys than in other organs, indicating an efficient D-luciferin targeting and release in vivo. The scale is in photons/sec/sm²/sr;

FIG. 18: Enhanced and prolonged anti-tumour effect of doxorubicin targeted by ferriliposomes:
Dynamics of the tumour volumes reduction after doxorubicin treatment (15 mg/kg) using a conventional systemic administration (Dox; ) or using the targeted ferriliposome system (Dox+FLt; ▴). Magnetic field strength was 0.33 T. Each value represents mean±SE of 4 trials. *, P<0.05 as compared to the Dox group by Student's t-test;

FIG. 19: Schematic of treatment design and treatment groups:

FIG. 19A: Primary tumour cells obtained from 14 week old MMTV-PyMT transgenic mice were culture-expanded and transplanted into the left inguinal mammary gland of a mouse (FVB/N mouse strain); FIG. 19B: Treatment by 10 intraperitoneal injections for all groups was performed every second day after tumour volume (Tv) reached 125 mm³;

FIG. 20: The anti-tumour effect of ferriliposome targeted cysteine proteases inhibitor JPM-565:
On the next day after the last injection mice were sacrificed and the final volumes of excised tumours were measured. Data are presented as mean±SE. Statistics were analyzed using Student's t-test;

FIG. 21: Expression of cysteine cathepsins after treatment with cysteine proteases inhibitor JPM-565:
Comparison of mRNA expression of cathepsin B, cathepsin L, cathepsin H and cathepsin X in tumour samples from different groups after treatment measured by RTQ-PCR. Data are presented as means±SE, n=3-4, expressed as a percentage of the control (100%);

FIG. 22: Activity of cysteine cathepsins in different organs after JPM-565 administration:
Liver, lungs, kidneys and pancreas were prepared 5 h after the final injection and cysteine cathepsin activity was measured by hydrolysis of the fluorogenic substrate Z-Phe-Arg-AMC. Data are presented as means and standard errors. *P<0.05 by t-test;

FIG. 23: In vivo detection of intraperitoneally administered ferriliposomes in intra-abdominal lymph nodes:
Fluorescence microscopy of the renal lymph node after intraperitoneally administrated Alexa Fluor 555™-functionalized ferriliposomes. Red fluorescence of Alexa Fluor 555™ indicates the residual presence of ferriliposomes in the lymphatic nodule without any major accumulation detected. The scale bar corresponds to 200 μm;

FIG. 24: Effect of JPM-565 treatment on proliferation of mammary carcinomas:
Proliferating cells were detected by immunohistochemical analysis based on the proliferation marker Ki67 (brown staining) Representative images are shown for control mice and mice treated with the cysteine cathepsin inhibitor JPM-565. The scale bar corresponds to 200 μm;

FIG. 25: Vascularization of mammary carcinomas after treatment with JPM-565:
Representative images of immunofluorescence staining of the endothelial cell specific marker CD31 (red staining). Rat anti-CD31 antibody (BD Pharmingen; 1:100 dilution) and secondary donkey anti-rat Cy3 antibody (Jackson ImmunoResearch; 1:200 dilution) were used for immunodetection of endothelial cells in cryopreserved tumour sections. The scale bar corresponds to 200 μm;

FIG. 26 Histogram for a size distribution of nanoparticles of oxide ferrimagnetics with spinel structure of according to formula: Co_{0.84}Fe_{2.16}O₄;

FIG. 27 Relaxation time (FIG. 27A) T₁ [symbol] (FIG. 27B) T₂ vs. concentration of oxide ferrimagnetics nanoparticles with spinel structure according to formula CO_{0.84}Fe_{2.16}O₄ in stabilizing buffer (a biocompatible saline solution);

FIG. 28 Magnetic resonance image (MR-image) of three samples, containing various concentrations nanoparticles of oxide ferrimagnetics with spinel structure according to formula: Co_{0.84}Fe_{2.16}O₄ in 1% agarose (1:1% agarose; 2: 0.017 MM; 3: 0.17 MM; 4: 1.7 MM, and 5: phantom containing CuSO₄.H₂O) and a dependence of contrast on relaxation time: FIG. 28A—T₁ MR-image for echo-time Tₑ=8.5 ms and relaxation time Tₚ=400 ms; FIG. 28B—T₂ MR-image for Tₑ=60 ms and Tₚ=2000 ms;

FIG. 29 Scheme of 1% agarose samples, one of which contains locally added oxide ferrimagnetics nanoparticles with spinel structure according to formula: CO_{0.84} Fe_{2.16}O₄ (FIG. 29A) and their transversal MR-images: FIG. 28B—T₁ MR-image for echo-time Tₑ=8.5 ms and relaxation time Tₚ=400 MC; FIG. 28C—T₂ MR-image for Tₑ=60 ms [symbol] Tₚ=2000 ms with their corresponding graphic profiles of MR signal;

FIG. 30 Sagittal MR-images: FIG. 30A—T₁ MR-image for echo-time Tₑ=8.5 ms and relaxation time Tₚ=400 MC; FIG. 30B—T₂ MR-image for Tₑ=60 ms [symbol] Tₚ=2000 ms with their corresponding graphic profiles of MR signal;

FIG. 31 Diagrammatic representation oxide ferrimagnetics with spinel structure nanoparticles according to formula: Co_{0.84}Fe_{2.16}O₄, encapsulated in liposome and hypothetic chemical agent (FIG. 30A) and atomic-force microscopy image (AFM image) of magneto-liposomes (FIG. 30B); and

FIG. 32 Magnetic resonance scanning of targeting magnetoliposomes (TE=60 ms, TR=2000 ms, slice thickness=1 mm) of transplanted mice mammary tumor in vivo: before the injection of 200 μl ferriliposome solution followed by magnetic field application and 24 hours later.

### REFERENCES

Ai H et al Magnetite-loaded polymeric micelles as ultrasensitive magnetic-resonance probes Advanced Materials 17 1949-+2005
Arrueboa M Fernández-Pachecoa R Ibarraa M R & Santamarí a S Magnetic nanoparticles for drug delivery Nanotoday 2 22-32 2007
Atanasijevic T Shusteff M Fam P & Jasanoff A Calcium-sensitive MRI contrast agents based on superparamagnetic iron oxide nanoparticles and calmodulin Proc Natl Acad Sci USA 103 14707-14712 2006
Babincova M Machova E 1999 Dextran enhances calcium-induced aggregation of phosphatidylserine liposomes possible implications for exocytosis Physiol Res 48 319-21
Bacri J Perzynski R Salin D Cabuil V Massart R J 1990 Ionic ferrofluids a crossing of chemistry and physics J Magn Magn Mat 85 27-32
Bell-McGuinn K Garfall A Bogyo M Hanahan D & Joyce J A Inhibition of cysteine cathepsin protease activity enhances chemotherapy regimens by decreasing tumor growth and invasiveness in a mouse model of multistage cancer Cancer Res 67 7378-7385 2007
Bogdanov A A Martin C Weissleder R & Brady T J Trapping of Dextran-Coated Colloids in Liposomes by Transient Binding to Aminophospholipid—Preparation of Ferrosomes Bba-Biomembranes 1193 212-218 1994
Bulte J W de Cuyper M Despres D & Frank J A Short- vs long-circulating magnetoliposomes as bone marrow-seeking MR contrast agents J Magn Reson Imaging 9 329-335 1999
Bulte J W M de Cuyper M Despres D & Frank J A Preparation relaxometry and biokinetics of PEGylated magnetoliposomes as MR contrast agent J Magn Magn Mater 194 204-209 1999
Bulte J W M et al Selective Mr Imaging of Labeled Human Peripheral-Blood Mononuclear-Cells by Liposome Mediated Incorporation of Dextran-Magnetite Particles Magnet Reson Med 29 32-37 1993
Ceelen W P & Flessner M F Intraperitoneal therapy for peritoneal tumors biophysics and clinical evidence Nat Rev Clin Oncol 7 108-115 2010
Cornell J A 1991 The fitting of Scheffe-type models for estimating solubilities of multisolvent systems J Biopharm Stat 2 303-329
Di Paolo D et al Liposome-mediated therapy of neuroblastoma Methods Enzymol 465 225-249 2009
Fortin-Ripoche J P et al Magnetic targeting of magnetoliposomes to solid tumors with MR imaging monitoring in mice feasibility Radiology 239 415-424 2005
Galanzha E I et al In vivo magnetic enrichment and multiplex photoacoustic detection of circulating tumour cells Nat Nanotechnol 4 855-860 2009
Gocheva V et al IL-4 induces cathepsin protease activity in tumor-associated macrophages to promote cancer growth and invasion Genes Dev 24 241-255 2010
Greenbaum D et al Chemical approaches for functionally probing the proteome Mol Cell Proteomics 1 60-68 2002
Greenbaum D Medzihradszky K F Burlingame A & Bogyo M Epoxide electrophiles as activity-dependent cysteine protease profiling and discovery tools Chem Biol 7 569-581 2000
Guy C T Cardiff R D & Muller W J Induction of mammary tumors by expression of polyomavirus middle T oncogene a trans-genic mouse model for metastatic disease Mol Cell Biol 12 954-961 1992
Joyce J A et al Cathepsin cysteine proteases are effectors of invasive growth and angiogenesis during multistage tumorigenesis Cancer Cell 5 443-453 2004
Kim J W Galanzha E I Shashkov E V Moon H M& Zharov V P Golden carbon nanotubes as multimodal photoacoustic and photothermal high-contrast molecular agents Nat Nanotechnol 4 688-694 2009
Lee J et al Artificially engineered magnetic nanoparticles for ultra-sensitive molecular imaging Nature Medcine 13 95-99 2007
Liotta L A & Kohn E C The microenvironment of the tumour-host interface Nature 411 375-379 2001
Lopez J A Gonzalez F Bonilla F A Zambrano G Gomez M E 2010 Synthesis and characterization of Fe3O4 magnetic nanofluid Revista Latinoamericana de Metalurgia y Materiales 30 60-66
Martina M S et al Generation of superparamagnetic liposomes revealed as highly efficient MRI contrast agents for in vivo imaging J Am Chem Soc 127 10676-10685 2005
Medarova Z Pham W Farrar C Petkova V & Moore A In vivo imaging of siRNA delivery and silencing in tumors Nat Med 13 372-377 2007
Mohamed M M & Sloane B F Cysteine cathepsins multifunctional enzymes in cancer Nat Rev Cancer 6 764-775 2006
Morais P C Santos J G Silveira L B Gansau C Buske N Nunes W C Sinnecker J P 2004 Susceptibility investigation of the nanoparticle coating-layer effect on the particle interaction in biocompatible magnetic fluids Journal of Magnetism and Magnetic Materials 272 2328-2329 DOI DOI 10 1016/j jmmm 2003 12 473
Morais P C Santos R L Pimenta A C M Azevedo R B Lima E C D 2006 Preparation and characterization of ultra-stable biocompatible magnetic fluids using citrate-coated cobalt ferrite nanoparticles Thin Solid Films 515 266-270 DOI DOI 10 1016/j tsf 2005 12 079
Mueller M M & Fusenig N E Friends or foes—bipolar effects of the tumour stroma in cancer Nat Rev Cancer 4 839-849 2004
Na H B et al Development of a T1 contrast agent for magnetic resonance imaging using MnO nanoparticles Angew Chem Int Ed Engl 46 5397-5401 2007
Naiden E et al Magnetic properties and structural parameters of nanosized oxide ferrimagnet powders produced by mechanochemical synthesis from salt solutions Physics of the solid state 5 891-900 2003
Naiden E Zhuravlev V Itin V Terekhova O Magaeva A Ivanov Y 2008 Magnetic properties and structural parameters of nanosized oxide ferrimagnet powders produced by mechanochemical synthesis from salt solutions Physics of the solid state 50 894-900
Namiki Y et al A novel magnetic crystal-lipid nanostructure for magnetically guided in vivo gene delivery Nat Nanotechnol 4 598-606 2009
Orive G Anitua E Pedraz J L Emerich D F 2009 Biomaterials for promoting brain protection repair and regeneration Nature Reviews Neuroscience 10 682-U47 DOI Doi 10 1038/Nm2685
Rosi N L & Mirkin C A Nanostructures in Biodiagnostics Chem Rev 105 1547-1562 2005
Rossi A Deveraux Q Turk B & Sali A Comprehensive search for cysteine cathepsins in the human genome Biol Chem 385 363-372 2004
Sadaghiani A M et al Design synthesis and evaluation of in vivo potency and selectivity of epoxysuccinyl-based inhibitors of papain-family cysteine proteases Chem Biol 14 499-511 2007
Santos A M Jung J Aziz N Kissil J L & Puré E Targeting fibroblast activation protein inhibits tumor stromagenesis and growth in mice J Clin Invest 119 3613-3625 2009
Schurigt U et al Trial of the cysteine cathepsin inhibitor JPM-OEt on early and advanced mammary cancer stages in the MMTV-PyMT-transgenic mouse model Biol Chem 389 1067-1074 2008
Seo W S et al FeCo/graphitic-shell nanocrystals as advanced magnetic-resonance-imaging and near-infrared agents Nature Materials 5 971-976 2006
Sevenich L et al Synergistic antitumor effects of combined cathepsin B and cathepsin Z deficiencies on breast cancer progression and metastasis in mice Proc Natl Acad Sci USA 107 2497-2502 2010
Shapiro M G Atanasij evic T Faas H Westmeyer G G & Jasanoff A Dynamic imaging with MRI contrast agents quantitative considerations Magn Reson Imaging 24 449-462 2006
Sloane B F et al Cathepsin B and tumor proteolysis contribution of the tumor microenvironment Semin Cancer Biol 15 149-157 2005
Stollfuss J C et al Rectal carcinoma high-spatial-resolution MR imaging and T2 quantification in rectal cancer specimens Radiology 241 132-141 2006
Torchilin V Multifunctional and stimuli-sensitive pharmaceutical nanocarriers Eur J Pharm Biopharm 71 431-444 2009
Torchilin V P et al Poly Ethylene Glycol on the Liposome Surface—on the Mechanism of Polymer-Coated Liposome Longevity Bba-Biomembranes 1195 11-20 1994
Vasiljeva O & Turk B Dual contrasting roles of cysteine cathepsins in cancer progression apoptosis versus tumour invasion Biochimie 90 380-386 2008
Vasiljeva O et al Emerging roles of cysteine cathepsins in disease and their potential as drug targets Curr Pharm Des 13 387-403 2007
Vasiljeva O et al Reduced tumour cell proliferation and delayed development of high-grade mammary carcinomas in cathepsin B-deficient mice Oncogene 27 4191-4199 2008
Vasiljeva O et al Tumor cell-derived and macrophage-derived cathepsin B promotes progression and lung metastasis of mammary cancer Cancer Res 66 5242-5250 2006
Vlaskou D et al Magnetic and Acoustically Active Lipospheres for Magnetically Targeted Nucleic Acid Delivery Adv Funct Mater 20 3881-3894 2010
Wender P A et al Real-time analysis of uptake and bioactivatable cleavage of luciferin-transporter conjugates in transgenic reporter mice Proc Natl Acad Sci USA 104 10340-10345 2007
Zhao M Josephson L Tang Y & Weissleder R Magnetic sensors for protease assays Angew Chem Int Ed Engl 42 1375-1378 2003

### EXAMPLES

### Development and Characterization of Ferriliposomes

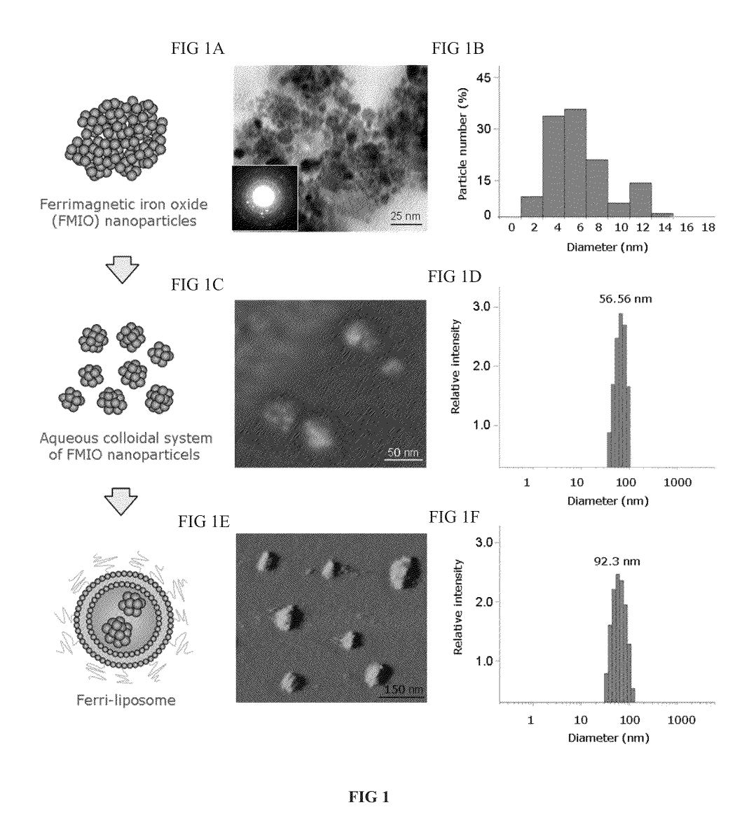

The iron oxide particles according to the present invention were prepared by mechanochemical synthesis using saline crystal hydrates (see Example 1). The use of saline crystal hydrates instead of conventional methods utilizing anhydrous salts changes the solid phase mechanism to soft mechanochemical synthesis in aqueous media, surprisingly resulting in a significantly increased reaction rate. Furthermore, this modification surprisingly resulted in ultrasmall spherical particles of 3-14 nm in diameter (>70% less than 8 nm) (FIGS. 1a, b).

The main limiting factor in using nanoparticles, in particular iron oxide nanoparticles in vivo is their low colloidal stability. The method of the present invention for preparing a biocompatible aqueous colloidal system prevents their agglomeration, leading to a more narrow nanoclusters particle size distribution (FIG. 1c; FIG. 6). The concentration of iron oxide nanoparticles was measured by flame atomic absorption spectrometry and a unit average size of nanoparticles was determined by dynamic light scattering (DLS) (FIG. 1d). The resulting iron oxide nanoparticles displayed a negative surface zeta potential of 27.9±4.3 mV at pH 7.4 and 37° C.

The suspension of the present invention surprisingly exhibits high colloidal stability under physiological conditions as well as at various pH values and ionic strengths (Example 1, FIG. 6).

Stabilized iron oxide nanoparticles were encapsulated in sterically stabilized polyethylene glycol (PEG) coated liposomes (PEGylated; Stealth liposomes), forming ferriliposomes of about 100 nm diameter. Modification of the liposome surface with PEG is known to greatly reduce the opsonization of liposomes and their subsequent clearance by the reticuloendothelial (mononuclear-phagocyte) system, resulting in a substantially prolonged circulation half-life. This was confirmed in a cellular experiment (FIG. 7). The liposomes loaded with iron oxide particles appeared, under atomic force microscopy (AFM), as spheroids with diameters of 0.09-0.11 μm (FIG. 1e), consistent with the average diameter of 92.3 nm measured for ferriliposomes by DLS (FIG. 1f). Because of their size, hydrophobic and hydrophilic character, biocompatibility, together with the internal hollow space (FIG. 7), the system of ferriliposomes of the present invention enables simultaneous encapsulation of iron oxide nanoparticles with other substances, such as pharmaceutical drugs or DNA, and their subsequent targeted delivery in the organism, in particular mammals, more preferably humans.

The oxide ferrimagnetics with spinel structure nanoparticles according to formula Co_{0.84}Fe_{2.16}O₄ were prepared by mechanochemical synthesis using saline crystal hydrates (see Example 2) with size distribution of nanoparticles (FIG. 26).

The size of spinel oxide ferrimagnetic nanoparticles in a suspension (aqueous colloidal system) was determined using a dynamic light scattering (Dynamic Light Scattering Detector PD 2000 DLS Plus). Ion concentration was determined by flame-spectroscopy using a Varian Spectr AA 110 atomic absorption spectrometer.

Characterization of MR Contrast Properties of Iron Oxide Nanoparticles In Vitro

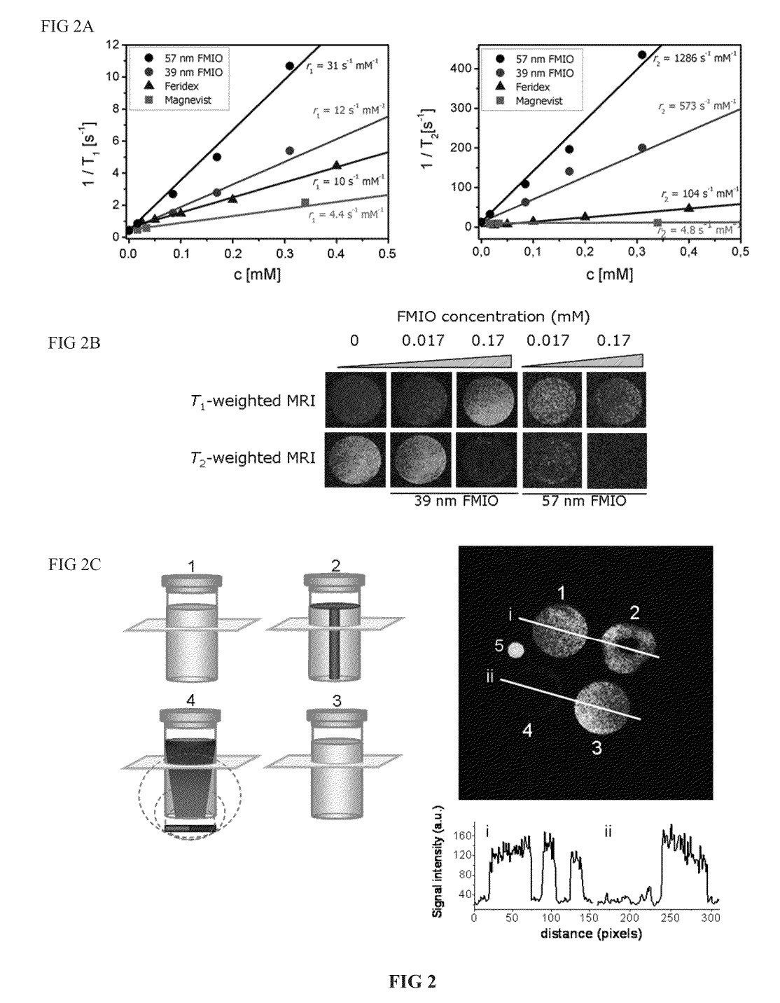

The MR contrast properties of the stabilized nanoparticle suspension in vitro have been evaluated in MRI agarose phantoms (MRAP), simulating the tumour tissue, using 1% agarose, with T₂≈80 ms, which are similar to those of tumour tissues. MRAPs containing iron oxide nanoparticles with nanoclusters mean hydrodynamic diameter of 39 nm and 57 nm, respectively, were screened for the MRI contrast properties. The longitudinal (T₁) and transverse (T₂) relaxation times were measured at different iron oxide nanoparticle concentrations, and r1 and r2 relaxivities were determined to be 12 s⁻¹ mM⁻¹ and 573 s⁻¹ mM⁻¹ for the 39 nm nanoparticles, and 31 s⁻¹ mM⁻¹ and 1286 s⁻¹ mM⁻¹ for the 57 nm iron oxide nanoparticles. Comparison with the commercially available SPIO nanoparticles Feridex® (Bayer HealthCare Pharmaceuticals) and the standard gadolinium-based T₁ contrast agent Magnevist® (Bayer HealthCare Pharmaceuticals) revealed that iron oxide nanoparticles exhibited several fold higher relaxivities than commercial MRI contrast agents, resulting in ultra-sensitive MRI detection (FIG. 2a). Moreover, a 20-70% improvement in the r₂ relaxivity was found when compared to the best iron oxide-based nanoparticles described in the literature. The high r₂ relaxivity, most probably attributed to clustering of the iron oxide nanoparticles, is supported by the enhancement of relaxivity of iron oxide nanoparticles with the higher hydrodynamic diameter of the clusters. This demonstrates iron oxide nanoparticles as high-performance MRI contrast agents, that enable extremely sensitive T₂-weighted MM measurements.

To verify the effectiveness of the iron oxide nanoparticles as positive T₁ and negative T₂ contrast agents, T₁-weighted and T₂-weighted images of the control phantom and MRAPs with 0.017 mM of the 39 nm and 0.17 mM of the 57 nm iron oxide nanoparticles were obtained (FIGS. 2b). The signal intensity of these phantoms was significantly diminished on the T₂-weighted MR scans, while the same concentration of iron oxide nanoparticles demonstrated enhanced MRI signal on the T₁-weighted images (FIG. 2b). Hence, unique simultaneous T₁ and T₂ MR contrast properties of iron oxide nanoparticles were demonstrated, enabling their use as single contrast agents for both T₁ and T₂-weighted MR scans, thereby enhancing the diagnostic properties of MR imaging. Moreover, the 2-fold higher sensitivity of the 57 nm iron oxide nanoparticles relative to that of the smaller 39 nm nanoclusters in T₂-weighted MR scans (FIG. 2b), suggests the former to be extremely effective contrast agents. However, in drug delivery applications the smaller 39 nm nanoclusters, with their still superior contrast properties, are preferable.

To demonstrate the targeting effectiveness of the magnetic nanoparticles suspension and their MM contrast properties upon internalization into the phantom, two techniques for iron oxide nanoparticle delivery into the MRAP were used: (i) direct injection of the iron oxide nanoparticle solution with an 0.3 mm needle into the centre of MRAP (sample 2 in FIG. 2c) and (ii) application of the iron oxide nanoparticle dispersion onto the MRAP surface by positioning the magnetic field (B=0.33 T) at the bottom of the vial (sample 4 in FIG. 2c). As seen on the T₂-weighted MR image and from the MR signal intensity profile, both delivery methods were effective, demonstrating a clear difference between the MR signal of the iron oxide nanoparticles and the MRAP. Moreover, the MR signal of sample 4 disappeared completely, indicating successful penetration of the iron oxide nanoparticles through the phantom matter as a result of targeting by an external magnetic field. Collectively, these results show that the iron oxide nanoparticles of the present invention can be used as driving components for multifunctional targeted delivery systems enabling simultaneous MR detection. In addition, the MM contrast properties of iron oxide nanoparticles remain the same after their encapsulation into the liposomes (FIG. 9), supporting the use of ferriliposomes in medical applications.

Characterization of MR contrast properties of cobalt oxide ferrimagnetic nanoparticles with spinel structure according to formula CO_{0.84} Fe_{2.16}O₄ in vitro

The longitudinal (T₁) and transverse (T₂) relaxation times were measured at different concentrations of cobalt oxide ferrimagnetics nanoparticles with spinel structure in 1% agarose at room temperature for the frequency ¹H [symbol] MP ν_{H}=100 MHz (FIG. 28). The spin-lattice relaxation time T1 was measured at a frequency of relaxation TR=15 s and the time 1400-15 between pulses. The spin-spin relaxation time T2 was measured by sequential spin-echo with repetition time TR=500 ms at different time of incoming echo. The echo time was 15 ms to 500 ms. Longitudinal (r1) and transverse (r2) determine the degree of relaxation at a rate r1=22 s−1 mM-1, r2=503 s−1 mM−1. The longitudinal (r1) and transverse (r2) relaxivities were determined to be 22 s⁻¹ mM⁻¹ and 503 s⁻¹ mM⁻¹, relatively. To verify the effectiveness of nanoparticles as positive T₁ and negative T₂ contrast agents a 2.35 T magnet was applied at the ambient temperature. T₁-weighted and T₂-weighted images of samples of agarose phantoms were obtained for various concentrations of nanoparticles using the following parameters of frequency spin eho: viewing area was 3 cm, thickness of the slides was 1 mm, matrix size was 256×256. The ratio TR/TE=60/2000 ms was used. A sample with a concentration of nanoparticles of 0.34 mM showed complete disappearance of the magnetic resonance (MR) signal (FIGS. 29, 30).

Also, the loss of signal was observed in the sample with the concentration of nanoparticles of 3.4 mm. The signal intensity for the concentration of 0.034 mM was comparable to that for 1% agarose. The results have demonstrated the possibility of using nanoparticles as contrast agents in selecting a desired concentration. The properties of oxide ferrimagnetics nanoparticles with spinel structure according to formula CO_{0.84} Fe_{2.16}O₄ as T1 and T2 contrast agents have also been demonstrated using a sample containing 1% agarose, which is locally added with a solution of 0.34 mM nanoparticles. The nanoparticles were injected into the middle of the agarose phantom (FIG. 30). On T2 MR image the negative contrast was observed in the same position within a sample. This confirms a contrast effect of spinel oxide ferrimagnetics nanoparticles at their concentration of 0.34 mM. MR scan parameters were the same as those mentioned above.

Efficacy of ferriliposomes as an MRI-visible drug delivery system in vivo

To establish the efficacy of the prepared ferriliposomes for in vivo applications a genetically engineered mouse model of human breast cancer (MMTV-PyMT) was employed resulting in a widespread transformation of the mammary epithelium and in the development of multifocal mammary adenocarcinomas. Initially, ferriliposomes were demonstrated to be non-cytotoxic in mouse embryonic fibroblasts (MEFs) and primary mouse tumour cells (see FIG. 10). Possible adverse effects of iron oxide nanoparticles were additionally evaluated in an acute toxicity experiment using rats. No significant differences in blood biochemistry and histopathological analysis were observed 7 and 12 days after administration between control animals and animals treated with 500 mg/kg iron oxide nanoparticles (see Table 1 and FIG. 11).

TABLE 1
Blood biochemistry after administration of iron oxide nanoparticles
in an acute toxicity study.

| | | Blood urea | Alanine | Aspartate | Creatine | Total |
|---|---|---|---|---|---|---|
| Mice | Creatinin | nitrogen | transaminase | transaminase | phosphokinase | bilirubin |
| groups | μg/l | mg/l | U/l | U/l | U/l | μg/l |
| control | 34.0 ± 0.82 | 4.18 ± 0.27 | 142.5 ± 6.31 | 170.5 ± 5.23 | 3471 + 96 | 7.2 + 0.31 |
| 7 days | 34.0 ± 0.83 | 5.81 ± 0.4 | 94.44 ± 4.38 | 178.4 ± 6.06 | 5151 + 331 | 8.7 + 0.45 |
| 12 days | 39.0 ± 0.87 | 4.11 ± 0.25 | 91.57 ± 3.41 | 205.3 ± 3.53 | 4010 + 264 | 8.3 + 0.61 |

Having shown that the system is suitable for in vivo applications, ferriliposomes were injected intraperitoneally into a MMTV-PyMT tumour bearing mouse, under a magnetic field applied for 1 hour to the first left inguinal mammary tumour. iron oxide nanoparticles delivered by ferriliposomes were detected as a dark area on the T₂-weighted MR images, 1 and 48 hours post injection (FIG. 3a, FIG. 12), confirming their successful targeting to the tumour region and their apparent MRI contrast effect. Furthermore, in addition to spreading through the tumour tissue, nanoparticles were detected in the tumour surroundings, the tumour microenvironment (FIG. 12). This ability of ferriliposomes could be of particular value for developing novel strategies to treat cancer, with the further advantage of being regulated by magnetic field (FIG. 13). The effectiveness of the system was confirmed by intravenous administration of ferriliposomes (FIG. 14). Collectively, these results demonstrate both the efficacy of ferriliposomes for magnetic field targeted drug delivery and the possibility of monitoring their distribution by noninvasive MRI technology. The intracellular delivery of targeted ferriliposomes was validated in tumour and stromal cells, using a fluorescent marker (Alexa Fluor 555™) as a model drug. The Alexa Fluor 555™-functionalized ferriliposome suspension was incubated for 3 hours with primary MMTV-PyMT tumour cells and MEFs. Fluorescence microscopy analysis revealed very efficient internalization of the Alexa Fluor 555™ by both types of cells (FIG. 3b). Moreover, compartmentalization of fluorescent particles in intracellular vesicles of primary tumour cells and fibroblasts provides clear evidence for successful endocytosis of the ferriliposome cargo. This carrier system therefore represents a promising candidate for targeted drug delivery into a tumour and its microenvironment, enabling more effective cancer therapy. To confirm in vivo the release of drug encapsulated in ferriliposomes, we crossed MMTVPyMT mice (PyMTtg/+) with the FVB/N mouse strain expressing firefly luciferase under the control of the β-actin promoter (FVB.luctg/+). The resulting double transgenic mice (FVB.luctg/+;PyMTtg/+) develop breast tumours with simultaneous expression of luciferase throughout the body. Twenty four hours after administration and targeting of ferriliposomes loaded with the luciferase substrate, D-luciferin, to the tumour, a luminescent signal was imaged exclusively in the tumour region (FIG. 3c), indicating effective release of the cargo. The efficiency of the system was also confirmed by intravenous administration of ferriliposomes (FIG. 15). Furthermore, nanoparticles were successfully excreted from the body without any evident accumulations (FIGS. 16, 17), which is another critical parameter for their in vivo application.

The efficacy of ferriliposomes as an MRI-visible drug delivery system for oxide ferrimagnetic nanoparticles with spinel structure according to formula Co_{0.84} Fe_{2.16}O₄ in vivo is shown in FIG. 32. The bright area of image of the magnet 0.3 T attaching point (white arrow) and those of T1-weighted MR scans as well as the corresponding dark areas of image of the magnet 0.3 T attaching point (dotted white arrow) on T2-weighted MRI scans evidence a preferential accumulation of magnitoliposoms confirming their applicability as both positive and negative MR contrast agents.

Ferriliposome Delivered JPM-565 Inhibits Growth of Mammary Tumour Lesions

The initial testing of the ferriliposome system for the targeted drug delivery was performed with a standard cancer chemotherapy drug, doxorubicin. Even a single dose treatment with doxorubicin targeted by ferriliposomes resulted in a 90% reduction of tumour volume two weeks after administration, compared with 60% decrease obtained by the standard doxorubicin administration (FIG. 18). In an independent approach, a compound ineffective due to the poor bioavailability was surprisingly converted into an effective one: For this purpose we selected JPM-565, a small molecule broad spectrum inhibitor of cysteine cathepsins, which was very potent in treatment of pancreatic islet cells cancer in a mouse model, but lacked any efficacy in the MMTV-PyMT mouse breast cancer model due to its very poor bioavailability, although genetic ablation of several cathepsins attenuated tumour progression in that model. Moreover, the cysteine cathepsins participating in multiple stages of tumour progression largely originate from the cells of the microenvironment, thereby offering the opportunity to simultaneously validate the novel concept of targeting tumour microenvironment and the novel drug delivery system in order to improve cancer treatment. In order to overcome the limitations of the transgenic MMTV-PyMT mouse model possessing multifocal mammary tumours which are difficult to follow, and to secure the functional immune system (as compared to the xenograft approach), an orthotopically transplanted mouse mammary tumour model was developed by inoculating 5×10⁵ primary MMTV-PyMT tumour cells into the mammary gland of the congenic immunocompetent recipient mouse (FVB/N mouse strain) (FIG. 19a). In contrast to the original transgenic model, the orthotopic transplanted model results in a single tumour that can be easily monitored due to the lower heterogeneity with regard to tumour latency and growth, thus making it an ideal model for drug efficacy studies. Starting with a tumour volume of 125 mm3, ferriliposomes containing JPM-565 at a concentration of 100 mg/kg were injected intraperitoneally 10 times every second day under a magnetic field focused on the tumour. Tumour sizes were measured the day after each injection. At the end of the treatment, tumours were excised and their volumes determined. The anti-tumour effect of non-loaded ferriliposomes and different therapeutic modalities and forms of JPM-565 were compared (FIG. 19b). Mice treated by targeted JPM-565 loaded ferriliposomes displayed a significant lag in tumour growth compared with all other groups (FIG. 4a), without any adverse effects. Furthermore, the volumes of the excised tumours in group treated by targeted JPM-565 loaded ferriliposomes were significantly smaller than in other groups (FIG. 4b and FIG. 20), suggesting a successful cathepsin inhibition in this group. This was confirmed by the substantial reduction of cysteine cathepsin activity measured exclusively in tumour samples from this group (FIG. 4c), whereas no difference in cathepsin expression was detected between all the groups (FIG. 21). In agreement with previous studies a significant inhibition of cysteine cathepsins was observed in the organs close to the peritoneum (FIG. 22). Subsequent clearance from the peritoneum through the lymph nodes was also confirmed (FIG. 23). To address the role of cysteine cathepsins in tumour biological processes, we investigated the effect of cathepsin inhibition on tumour proliferation, vascularisation and invasiveness. Cell proliferation was quantified by immunohistochemical detection of the proliferation marker Ki67, revealing a significant decrease in proliferation rate of tumours treated with targeted JPM-565 as compared to the other groups (FIG. 4d and FIG. 19), corroborating reduced tumour growth in that cohort of mice. Based on the distribution of the endothelial cell marker CD31, no difference in vascularisation of the tumour samples was observed upon treatment (FIG. 20). However, there was a trend of translocation of the cell-adhesion protein E-cadherin from the cytosol to the cell surface following treatment with targeted JPM-565 (FIG. 4e), resulting in decreased invasiveness and progression of cancer. To confirm the targeting of JPM-565 to the tumour, the treatment scheme was mimicked by loading ferriliposomes with a fluorescent marker (Alexa Fluor 546™). Evidently, these ferriliposomes were successfully targeted to the tumour site, and uptake of their content by cells of the tumour microenvironment was clearly established (FIG. 5a). Moreover, it was demonstrated in vivo that the marker is compartmentalized in the intracellular vesicles of tumour stroma cells (FIG. 5a, insert). The latter is of particular importance since cathepsins from tumour stroma are believed to play an important role in the processes leading to tumour progression.

Although intraperitoneal administration of therapeutic agents is an important adjunct to surgery and systemic chemotherapy of cancer in selected patients 44 it was evaluated the effectiveness of intravenous administration of the delivery system for targeting a tumour and its microenvironment in MMTV-PyMT transgenic female mouse. The fluorescence of ferriliposome cargo (Alexa Fluor 555™) was found to be co-localized both with the stroma (FIG. 5b, CD206 marker for tumour-associated macrophages) and the tumour cells (FIG. 5c, epithelial marker E-cadherin) in the targeted PyMT tumour tissue. These results clearly demonstrate the potential applicability of the ferriliposomes in various therapeutic scenarios.

### Example 1

### Synthesis of Iron Oxide Nanoparticles According to the Present Invention

The iron oxide nanoparticles synthesized are preferably ferrimagnetic (magnetic ferrite spinel, magnetite, Fe₃O₄) and are also called FMIO (ferrimagnetic iron oxide nanoparticles). Ferrimagnetic iron oxide (iron oxide) nanoparticles (magnetite, Fe₃O₄) were manufactured by modified and optimised mechano-chemical synthesis. The standard mechanochemical synthesis of iron oxide nanoparticles is for example described in Naiden et al., 2003. However, according to the present invention, saline crystal hydrates are used for the first time for generating iron oxide nanoparticles. In particular, the salt crystal hydrates FeSO₄. 7 H₂O and FeCl₃. 6H₂O were used. In order to prevent heating of the reagent mixture during activation, we additionally introduced sodium chloride as an inert component in the ratio 1:2. The mechanochemical synthesis was performed in an MPV planetary mill at 60 g acceleration and the weight ratio of the powder (i.e. the reaction mixture comprising the two salt crystal hydrates and NaOH) and balls was 1:20. The reaction in the planetary mill was performed for 30 min. The obtained product was washed on a filter with distilled water until the salts were completely removed. The electron microscopy and size distribution of iron oxide nanoparticles are shown in FIG. 1. It could be shown, that iron oxide nanoparticles with a narrow and adequate size distribution were generated. The use of saline crystal hydrates instead of conventional methods utilizing anhydrous salts surprisingly changes the solid phase mechanism to soft mechanochemical synthesis in aqueous media, resulting in a significantly increased reaction rate. Furthermore, this modification surprisingly resulted in ultrasmall spherical particles of 3-14 nm in diameter (>70% less than 8 nm).

### Example 2

### Synthesis of Oxide Ferrimagnetics Nanoparticles with Spinel Structure According to Formula CoₓFe₃₋ₓO₄, Wherein 0.1≦x≦0.99 According to the Present Invention

The oxide ferrimagnetics nanoparticles with spinel structure were obtained by mechanochemical synthesis using iron and cobalt chlorides as basic reagents in the presence of sodium chloride as inert component according to the following reaction:

2FeCl₃+CoCl₂+Ca(OH)₂+3Na₂CO₃=CoₓFe₃₋ₓO₄+CaCl₂+6NaCl+3CO₂↑+H₂O

where 0.1≦x≦0.99.

The value of 0.6≦x≦0.98 is preferable.

The starting materials used for the synthesis were FeCl₃, CoCl₂ in the form of salt crystal hydrates. For the purpose of changing the ratio of cobalt and iron in the end product and preventing the heating of the mixture of reagents and the aggregation of nanoparticles sodium chloride as additional inert component was injected. The mixture was sealed in a hardened steel drums with steel balls with a diameter of 4-5 mm. Mechanochemical synthesis was carried out in a MPV planetary mill with the acceleration of 55-60 g.

The conditions for accomplishment of a given technical effect of the invention are the strict adherence to the weight ratios of the mass of reaction mixture to the mass of inert component of 1:(1÷4) and the mass of powder to the mass of balls equal to 1:20, and the time for performing of mechanochemical synthesis of 10÷60 min. The product obtained through a heat treatment at 100° C. for 0.5÷1:00 (or without treatment) was washed in the filter with distilled water until free of salts and dried at room temperature, and then, if necessary, sonicated and centrifuged (UZDN-2T and <<Bekman J2-21″).

Phase composition, morphology, dispersion, and structural parameters of nanoparticles were determined by X-ray diffraction (XRD) using the Schimadzu XRD-6000 device with CuKα-radiation and by transmission electron microscopy (TEM) using the EM-125 device. The specific surface area (S) was determined by the method of thermal desorption of nitrogen (‘SORBI’ N 4.1) and the chemical composition was analyzed by X-ray fluorescence analysis (XRF) using Schimadzu XRD-1800 device and by inductively coupled plasma-atomic emission spectrometry (ICP-AES) using iCAP-6300 Duo, Thermo Scientific spectrometer. The data of X-ray structure analysis were processed using the full-profile analysis program POWDER CELL 2.5. The average diameter of particles was calculated from the values of specific surface area and particle density.

In the study of magnetic properties of ferrite spinel according invention used methods for analyzing the temperature dependence of initial magnetic permeability at a frequency of 10 kHz, and the magnetization curves and their derivatives are obtained in pulsed magnetic fields up to 3 T by the method described in (V. U. Kreslin, E. P. Naiden/PTE, 2001, No. 5, p. 63).

The investigation of the end products of mechanochemical synthesis showed that the powder consists of nanosized spherical particles with a diameter of 3-20 nm which are loosely coupled with each other (FIG. 26). According to conditions the final product contains 60-96% vol. of cobalt spinel ferrite of cubic syngony and the other phases (Tables II, III).

Table II. Effect of activation time on the chemical and phase composition of oxide ferrimagnetic nanoparticles with spinel structure (CoₓFe₃₋ₓO4) (acceleration is 60 g, the mass of the balls: the mass of the reacting mixture=20:1, the mass of NaCl: the mass of the reacting mixture=2:1 and FeCl₃.6H₂O, CoCl₂.6H₂O are reagents).

TABLE III
Effect of ratio of the mass of reaction mixture to the mass of inert diluent
(NaCl) on the chemical and phase compositions of oxide ferrimagnetics
nanoparticles with spinel structure (CoₓFe₃₋ₓO4) (the acceleration is 60 g,
time of mechanochemical synthesis is 30 min., the mass of the balls:the
mass of the reacting mixture = 20:1, FeCl₃•6H₂O, CoCl₂•6H₂O
are reagents.

| Mechanoactivation | | |
|---|---|---|
| time, min | Chemical formula | Phase composition |
| 5 | Co_{0.71}Fe_{2.29}O₄ | spinel - 35% |
| | | Fe₂O₃ - 11% |
| | | β-FeOOH - 50% |
| | | amorph - 4% |
| 10 | Co_{0.66}Fe_{2.34}O₄ | spinel - 60% |
| | | Fe₂O₃ - 7% |
| | | β-FeOOH - 27% |
| | | amorph - 4% |
| 15 | Co_{0.67}Fe_{2.33}O₄ | spinel - 77% |
| | | Fe₂O₃ - 4% |
| | | β-FeOOH - 15% |
| | | amorph - 4% |
| 25 | Co_{0.70}Fe_{2.30}O₄ | spinel - 94% |
| | | Fe₂O₃ - 1% |
| | | β-FeOOH - 1% |
| | | amorph - 2% |
| 30 | Co_{0.69}Fe_{2.31}O₄ | spinel - 95% |
| | | Fe₂O₃ - 1% |
| | | β-FeOOH - 0% |
| | | amorph - 4% |
| 40 | Co_{0.69}Fe_{2.31}O₄ | spinel - 96% |
| | | Fe₂O₃ - 1% |
| | | β-FeOOH - 0% |
| | | amorph - 3% |
| 50 | Co_{0.71}Fe_{2.29}O₄ | spinel - 87% |
| | | Fe₂O₃ - 1% |
| | | β-FeOOH - 11% |
| | | amorph - 4% |
| 60 | Co_{0.75}Fe_{2.25}O₄ | spinel - 85% |
| | | Fe₂O₃ - 0% |
| | | β-FeOOH - 14% |
| | | amorph - 3% |
| Ratio of the mass | | |
| of reaction mixture | | |
| to the mass NaCl | | Phase composition, |
| of inert diluent | Chemical formula | % vol. |
| 1:1 | Co_{0.8}Fe_{2.16}O₄ | spinel - 57.96% |
| | | Fe₂O₃ - 12.48% |
| | | β-FeOOH - 29.56 % |
| 1:2 | Co_{0.84}Fe_{2.16}O₄ | spinel - 91.25% |
| | | Fe₂O₃ - 3.56% |
| | | β-FeOOH - 15.19% |
| | | amorph - 0.02% |
| 1:3 | Co_{0.98}Fe_{2.02}O₄ | spinel - 84.53% |
| | | Fe₂O₃ - 7.4% |
| | | β-FeOOH - 8.07% |
| 1:4 | Co_{0.99}Fe_{2.01}O₄ | spinel - 90.22% |
| | | Fe₂O₃ - 3.19% |
| | | β-FeOOH - 6.59% |

The specific surface area of the resulting nanoparticles of cobalt spinel ferrite was 113 m²/g and the specific saturation magnetization was G.22-25 cm³/g.

The increase in the content of inert diluent from 1:2 to 1: (3 or 4) for the time of mechanochemical synthesis of 30 min results in the product of a nearly stoichiometric composition (Co_{0.99}Fe_{2.01}O₄) instead of the product with the chemical formula CO_{0.84}Fe_{2.16}O₄ (Table III).

With further increase in the content of inert component in the reaction mixture to 5:1 the yield of the end product significantly reduced. In this regard, the lower boundary value of the ratio of mass of reaction mixture to the mass of inert component was assumed to be 1:3.

The yield of the final product having chemical composition CoₓFe₃₋ₓO₄ was also determined by the conditions of mechanochemical synthesis. For the mass ratio of balls or sodium chloride to the mass of the reaction mixture of 20:1 and 2:1 respectively, the time of mechanical activation of 5 min. or less, and low conversion degree the yield of the end product was very low (35%) respectively (Table II).

With further increase in the duration of mechanical activation to 10 min the yield of the end product increased to 60% (lower value), and after treatment for 25÷40 min the yield was 94-96% (upper value). As a result, the value of the ratio of the mass of reaction mixture to the mass of inert component 1:2 was taken as the upper limit value. With increasing time of mechanical treatment the time of mechanical activation of 60 min was determined by a decrease in yield of the end product to 85%, other conditions being the same. The further increase in time of the mechanochemical process did not influence significantly the final product yield.

Thus, it is advisable to carry out the process of mechanical activation in the range of following parameters: the ratio of the mass of sodium chloride to the mass of the reaction mixture of (2:1)÷(3:1) and the time of mechanical activation of 10÷60 min. The heat treatment of the product of mechanochemical activation at 100±20° C. during 0.5÷1 h helps to ensure a final product having chemical composition CoₓFe₃₋ₓO₄, where 0.1≦x≦0.99 and which reveals high contrast properties at T₁ and T₂ relaxation times as shown below.

### Example 3

### Synthesis of Oxide Ferrimagnetics Nanoparticles with Spinel Structure of Formula MnₓFe₃₋ₓO₄, Wherein 0.1≦x≦0.99 According to the Present Invention

The investigation of the final products of mechanochemical synthesis shows that the manganese cubic spinel ferrite powder consists of nanosized spherical particles with a diameter range of 5-19 nm.

Depending on the synthesis conditions, the final products could contain to 90-99% vol. of spinel phase and the rest is mostly β-FeOOH and hematite.

As X-ray fluorescence analysis reveals that the chemical composition of nanosized manganese cubic spinel ferrite powder obtained at a ratio of the mass of reaction mixture to the mass of NaCl inert component 1:2 and at the time of the mechanochemical synthesis of 30-40 min has chemical formula MnₓFe₃₋ₓO₄, where x=0.50-0.96 and differs noticeably from stoichiometric composition. The increase in the content of inert diluent to 1:4 allows the obtaining of powders of nearly stoichiometric composition (Table IV).

The magnetic properties of nanosized powders of cubic spinel ferrites whose specific magnetization is 20-30 Gs·cm³/g determine their ability to be controlled by magnet.

TABLE IV
Chemical and phase composition of the end product vs. the ratio of the
mass of reagents to the mass of NaCl inert diluent (mass of the balls:
mass of the reacting mixture =20:1, activation time 30 min., heat
treatment for 1 h at 100° C.)

| Ratio of the mass | | |
|---|---|---|
| of reaction mixture | | Phase |
| to the mass of NaCl | | composition, |
| inert diluent | Chemical formula | % vol. |
| 1:1 | Mn_{0.62}Fe_{2.38}O₄ | spinel - 81.4 |
| | | Fe₂O₃ - 6.1 |
| | | β -FeOOH - 12.5 |
| 1:2 | Mn_{0.70}Fe_{2.30}O₄ | spinel - 93.7% |
| | | Fe₂O₃ - 2.4% |
| | | β-FeOOH - 3.9% |
| 1:3 | Mn_{0.84}Fe_{2.16}O₄ | spinel [symbol] - 98.2 |
| | | Fe₂O₃ - 1.8 |
| 1:4 | Mn_{1.04}Fe_{1..96}O₄ | spinel - 99.8 |
| | | Fe₂O₃ - 0.2 |

### Example 4

### Stabilization of Iron Oxide Nanoparticles According to the Present Invention

Iron oxide nanoparticles of Example 1 were suspended in a stabilizing buffer (20 mM sodium citrate buffer pH 7.4, containing 108 mM NaCl, 10 mM HEPES), sonicated with an ultrasonic disintegrator at 20 kHz, 3 min (Ultrasonic disintegrator, Branson) and centrifuged at 500 g for 3 min to separate the remaining undisrupted agglomerates. The resulting stable colloidal dispersion of non-aggregating nanoparticle clusters was characterized using flame atomic absorption spectrometry on a Varian SpectrAA 110 atomic absorption spectrometer (Varian, Mulgrave, Australia), dynamic light scattering (DLS) using a PDDLS/BatchPlus System (Precision Detectors), and field emission gun scanning electron microscopy (FEG-SEM) using an FESEM SUPRA 35 VP (Carl Zeiss) equipped with energy dispersive spectroscopy Inca 400 (Oxford Instruments). The zeta potential of iron oxide nanoparticles was measured by PALS Zeta Potential Analyzer Ver. 3.19 at pH 7.4 and 37° C. The result is shown in FIGS. 1a,b.

### Example 5

### Colloidal Stability of Iron Oxide Nanoparticles

Iron oxide nanoparticles were suspended in a stabilizing buffer (20 mM sodium citrate buffer pH 7.4, containing 108 mM NaCl, 10 mM HEPES). The resulting agglomerates were disrupted with an ultrasonic disintegrator (Branson), followed by separation of the remaining agglomerates by centrifugation at 500 g for 3 min (Eppendorf Centrifuge 5417C, Eppendorf). The nanoparticle drying step used in the initial procedure (Naiden, et al., 2003) was omitted. The colloidal stability of iron oxide nanoparticles was tested by increasing the ionic strength of the solution (NaCl concentration from 108 mM to 324 mM), and at two different pH values (pH 5.5, pH 9.0) of the solution. The colloidal stability was assessed by measurements of iron oxide cluster average sizes in the resulting suspensions by dynamic light scattering (DLS), using a PDDLS/BatchPlus System (Precision Detectors). It could be shown, that also stable suspensions could be obtained using these buffers.

### Example 6

### Colloidal Stability of Oxide Ferrimagnetics Nanoparticles with Spinel Structure According to Formula CO_{0.84}Fe_{2.16}O₄

A suspension of nanoparticles of CO_{0.84} Fe_{2.16}O₄ in a stabilizing buffer was obtained (pH=7) via ultrasonic disintegration (Bandelin). For the preparation of nanosuspension, for example, 100 mg of nanoparticles were dissolved in a stabilizing buffer (20 mM sodium citrate, 108 mM NaCl, 10 mM HEPES (4-(2-hydroxyethyl)-1-piperazinethan sulfonic acid) and sonificated (20 kHz, 50 V) for 5 min. In the course of sonification the large aggregates of nanoparticles were broken and covered with macromolecules of sodium citrate to prevent a reverse aggregation. Unbroken particles were precipitated in the gravitational field of 500 g.

### Example 7

### Synthesis of Ferriliposomes as an Example of Carrier Comprising Oxide Ferrimagnetic Nanoparticles with Spinel Structure

Iron oxide nanoparticles loaded liposomes (ferriliposomes) were prepared from 95% L-a-phosphatidylcholine (Avanti Lipids) and 5% 1,2-distearoyl-sn-glycero-3-phosphoethanolamine-N-[methoxy(polyethylene glycol)-2000] (Avanti Lipids) with a total lipid concentration of 2.75 mM. Organic solvent was evaporated in an Eppendorf Concentrator 5301 (Eppendorf), resulting in formation of dry lipid films. Their subsequent hydration with iron oxide nanoparticles in 20 mM citrate buffer pH 7.4, containing 108 mM NaCl and 10 mM HEPES, led to multilamellar vesicles containing nanoparticles. The multilamellar vesicles were extruded by mini-extruder containing a polycarbonate membrane, pore size 100 nm (Avanti Lipids), in order to generate nanosized unilamellar bilayer liposomes forming ferriliposomes. Non-encapsulated nanoparticles were removed by gel filtration on Sephadex™ G-25 M PD-10 columns (GE Healthcare). Ferriliposomes were separated magnetically from empty liposomes on a Dynal MPC-S magnetic separator (Dynal) and resuspended in stabilizing buffer. The morphology and size of the ferriliposomes was followed by atomic force microscopy (images were obtained with a Nanoscope III Multimode scanning probe microscope (Digital Instruments) operated in tapping mode) and DLS.

For fluorescence studies, ferriliposomes were functionalized with Alexa Fluor 546™-labelled dextran (Invitrogen) or non-conjugated Alexa Fluor 555™ (Invitrogen). Alexa Fluor 546™ or Alexa Fluor 555™ were suspended (100 μg/ml) in iron oxide nanoparticles containing the stabilizing buffer, and encapsulated in PEGylated liposomes as described. The fluorescent ferriliposomes were separated from non-encapsulated Alexa Fluor dye by gel filtration on a Sephadex™ G-25 M column (GE Healthcare).

Liposomes (ferriliposomes) loaded with oxide ferrimagnetic nanoparticles with spinel structure according to formula: Co_{0.84} Fe_{2.16}O₄ were prepared. An aliquot of lipids (2.6 mM of phosphatidylcholine and 0.2 mM of 0.2-distearol-sn-glycero-3-phosphoethanolamine-N-[methoxy(polyethylene glycol)-2000] (Avanti Polar Lipids Inc.), dissolved in chloroform was placed in a vacuum for the evaporation of the solvent with the formation of dry lipid films. Dry films were hydrated by adding of stabilized nanoparticles (3.4 mM).

The dispersion was emulsified by sonication in an ultrasonic bath for 5 min. The size of liposomes was determined using a dynamic light scattering (Dynamic Light Scattering Detector PD 2000 DLS Plus). Liposomal spheroids with a diameter of 90-110 nm were visualized by atomic force microscopy (AFM).

### Example 8

### Animal Models

Female FVB/N and FVB/N-TgN(MMTVPyVT)634Mu1 mice were used in accordance with protocols approved by the Veterinary Administration of the Republic of Slovenia (VARS) and the government Ethical Committee. Procedures for animal care and use were in accordance with the “PHS Policy on Human Care and Use of Laboratory Animals” and the “Guide for the Care and Use of Laboratory Animals” (NIH publication 86-23, 1996). In order to generate tumours for the treatment study, primary MMTV-PyMT tumour cells were obtained from 14 week old MMTV-PyMT transgenic mice as described 36, culture-expanded, suspended in 200 μl serum free Dulbecco's Modified Eagle Medium (DMEM) (Invitrogen), and 5×10⁵ cells were injected into the left inguinal mammary gland of the recipient mouse (FVB/N mouse strain).

### Example 9

### In Vitro and In Vivo MR Imaging

All MR experiments were performed on a TecMag Apollo MRI spectrometer with a superconducting 2.35 T horizontal bore magnet (Oxford Instruments) using a 25 mm saddle-shaped Bruker RF coil. Spin-lattice and spin-spin relaxation times (T₁ and T₂) were measured for different concentrations of iron oxide nanoparticles in 1% agarose at room temperature, using inversion recovery and spin-echo techniques, respectively. The longitudinal (rₜ) and transverse (r₂) relaxivities were calculated from rᵢ=(1/Tᵢ−1/Tᵢ₀)/c, where c is the concentration of iron oxide nanoparticles in mM, Tᵢ is the relaxation time at concentration c, Tᵢ₀ the relaxation time of 1% agarose, and i=1, 2 for T₁ and T₂. 2D MR images were taken using a standard multi-echo pulse sequence with an echo time (TE) of 8.5 ms and a repetition time (TR) of 400 ms for T₁ MR images, and with TE=60 ms and TR=2000 ms for T₂-weighted MR images. The field of view was 40 mm with an in-plane resolution of 156 μm and a slice thickness of 1 mm. Ferriliposomes were detected ex vivo by taking T₂-weighted MR images before and after injection of 50 μl ferriliposome solution (3.4 mM nanoparticles) into one of the tumours. For in vivo detection, an external magnet of 0.33 T (diameter 4.5 mm) was glued to the right inguinal mammary gland of 12 weeks old mouse by cyanoacrylate and 200 μl of ferriliposomes (3.4 mM nanoparticles) were intraperitoneally injected. The magnet was removed 1 hour after ablution with acetone. T₂-weighted MR images were taken before injection, 1 hour post-injection and 48 hours post-injection of ferriliposomes. During imaging, the mouse was anaesthetized by subcutaneous injection of ketamine/xylazine/acepromazine (50/10/1.0 mg/kg).

### Example 10

### Cell Culture and Assessment of Ferriliposome Internalization Ex Vivo

Primary MMTVPyMT cells were isolated and cultured as described 36. Mouse embryonic fibroblasts (MEFs) were generated from 12.5 days post-coitum mouse embryos of FVB/N mice; only low passage number cells (<4 passages total) were used for experiments. All primary cells were maintained in DMEM supplemented with 10% fetal bovine serum (Sigma), 2 mM L-glutamine (Invitrogen), 100 units of penicillin and 100 μg/ml streptomycin (Invitrogen). Cultured cells were maintained at 37° C. in a humidified 5% CO2 atmosphere. For fluorescence microscopy studies, primary MMTV-PyMT tumour cells and fibroblasts were cultured with 500 μl of Alexa Fluor 555™-functionalized ferriliposomes in normal culture medium on Lab-Tek™ Chamber Slides (Nunc). After incubation with nanoparticles for 3 hours, cells were washed with PBS, stained with Hoechst 33342 (Fluka) and examined with an Olympus fluorescence microscope (Olympus IX 81) with Imaging Software for Life Science Microscopy Cellf.

### Example 11

### Assessment of Ferriliposome Targeting and Internalization in Vivo by Bioluminescence

Female FVB/N-TgN(MMTVPyVT)634Mul mice (PyMTtg/+) developing multifocal adenocarcinomas were crossed with the FVB/N mouse strain expressing firefly luciferase under the control of the β-actin promoter (FVB.luctg/+)30. Resulting double transgenic mice (FVB.luctg/+;PyMTtg/+) develops breast tumours with simultaneous expression of luciferase through the whole body. For in vivo control of ferriliposomes distribution and content release, ferriliposomes were functionalized with D-luciferin (Sigma) by suspending in nanoparticles containing stabilizing buffer (2.5 mg/ml), followed by encapsulation in PEGylated liposomes. 400 μl of ferriliposomes loaded with D-luciferin (30 mg/kg) were intraperitoneally administered to the 10 weeks old FVB.luctg/+;PyMTtg/+ mouse and a magnet was attached to the 1st right pectoral mammary tumour. In the control experiment magnet was omitted. 24 hours after ferriliposomes administration magnet was detached and mice were imaged non-invasively by IVIS® Imaging System (integration time 5 minutes, IVIS® 100 Series). During the scan mice were kept under gaseous anaesthesia (5% isofluorane) and at 37° C. Due to the luciferase present in all cells, D-luciferin release and its subsequent conversion by luciferase resulted in emission of a bioluminescent signal that could be imaged with an IVIS® Imaging System.

### Example 12

### Treatment Study

The dosing regimen for JPM-565 treatment was determined based on previous reports and studies on RIP1-Tag2 and MMTV-PyMT mouse models 33-35, 45. JPM-565 had no discernable toxic side effects in the animal trials 33, 45. The regular injections, followed by magnetic targeting, were started when tumours reached a volume of 125 mm³. JPM-565 was dissolved in iron oxide nanoparticles containing stabilizing buffer and then encapsulated into PEGylated liposomes. The ferriliposomes loaded with JPM-565 were administered at a dose of 100 mg/kg every second day in 10 intraperitoneal injections (JPM+FL, n=9). Magnets of 0.33 T (diameter 4.5 mm) were attached to the tumour before the first injection and removed 24 hours after the last injection as described above. Control groups were treated with stabilizing buffer (Control, n=7), ferriliposomes with magnetic targeting (FLt, n=5), JPM-565 in nanoparticle stabilizing buffer (JPM, n=7) and ferriliposomes containing JPM-565 without magnetic targeting (JPM+FL, n=7). The horizontal and vertical tumour diameters were measured by digital calliper every second day until the end of treatment and volume was calculated using the formula V=(a×b2)π/6 where a and b are the longer and shorter diameter of the tumour. On the next day after the last injection mice were sacrificed and the excised tumour volumes calculated.

### Example 13

### Fluorescence Analysis of Ferriliposome Targeted Delivery and Internalization In Vivo

For the in vivo study, 200 μl of Alexa Fluor 546™ functionalized ferriliposomes were daily injected intraperitoneally to the orthotopic allograft breast cancer mouse model for 3 days. Alexa Fluor 555™ functionalized ferriliposomes were daily injected intravenously into the PyMT transgenic breast cancer mouse model for 2 days. A magnetic field was applied to the tumour for 12 hours immediately after each injection. On the next day after the last injection mice were sacrificed and the corresponding tumours were resected, fixed in 10% formalin overnight, dehydrated using Shandon Tissue Processor (Shandon Citadel 1000) and moulded with paraffin (Microm EC 350 Paraffin Embedding Station) or cryopreserved by snap freezing in liquid nitrogen. Paraffin sections were cut with 5 μm thickness, mounted with anti-fade media containing DAPI (Prolong@ Gold antifade reagent with DAPI, Invitrogen) and visualized as described above.

### Example 14

### Immunohistochemistry

Histological measurement of proliferation by Ki67 staining, and tumour vascularisation rate by CD31, were performed on frozen tissue slides. For data assessment, 10 fields per tumour were randomly selected using a 40× objective and quantified using TissueQuest software (TissueGnostics). Rabbit anti-mouse E-cadherin (Abeam; 1:100 dilution) and secondary antibody goat anti-rabbit Alexa Fluor™ 488 (Invitrogen; 1:100 dilution) were used for immunodetection of cell-adhesion protein E-cadherin on cryopreserved tumour sections. Rat anti-mouse monoclonal FITC conjugated CD206 (1:100; AbD Serotec) were used for the detection of tumour associated macrophages on cryopreserved tumour sections. Samples were co-stained with Hoechst 33342 (5 μg/ml, Fluka) and mounted in ProLong® Gold antifade reagent (Invitrogen) and examined with an Olympus fluorescence microscope (Olympus IX 81) with Imaging Software for Life Science Microscopy Cellf.

### Example 15

### Statistical Analysis

Quantitative data are presented as means plus/minus standard error. The differences of the JPM-565 treatment effect were compared using Student's t-test. When P-values were 0.05 or less, differences were considered statistically significant.

### Example 16

### Effect of liposome PEGylation on macrophage uptake

THP-1 monocytic cell line was grown at 37° C. in a humidified air atmosphere with 5% CO2. Cells were cultured in the RPMI 1640 medium, supplemented with 10% fetal calf serum, 2 mM L-glutamine, 100 U/ml penicillin, 100 μg/ml streptomycin and 10 mM HEPES buffer. THP-1 cells were differentiated into macrophages by the addition of 30 μl of 10 μM phorbol 12-myristate 13-acetate (PMA) for 24 hours. Cells were then incubated with 100 μl aliquots of Alexa Fluor 546™ functionalized PEGylated and non-PEGylated liposomes in Dulbecco's Modified Eagle Medium (DMEM) on 96-well optical bottom plate (Nunc, USA) for 15 minutes. In the next step, cells were washed 3 times with the phosphate buffer, pH 7.4, and examined in a 96-well Saphire (TECAN, Austria) plate reader at excitation and emission wavelengths of 561 nm and 572 nm, respectively.

### Example 17

### In Vitro Toxicity Assay

Mouse embryonic fibroblasts (MEFs) and primary MMTV-PyMT cells were maintained in DMEM supplemented with 10% fetal bovine serum (Sigma), 2 mM L-glutamine (Invitrogen), 100 units of penicillin and 100 μg/ml streptomycin (Invitrogen) at 37° C. in a humidified 5% CO2 atmosphere. For in vitro toxicity assay cells were incubated with ferriliposomes containing 3.4 mM iron oxide nanoparticles, or 3.4 mM and 55 mM iron oxide nanoparticles, in phosphate buffer, pH 7.4, for 24 hours at 37° C. In the control experiment nanoparticles were omitted. Exposure to phosphatidylserine and DNA fragmentation were measured by labelling cells with Annexin V-PE in the presence of propidium iodide according to the manufacturer's instructions. Cells were then subjected to flow cytometry (FACS) analysis using FACScalibur flow cytometer (Becton Dickinson, USA) and CellQuest software.

### Example 18

### Doxorubicin Treatment

Treatment study was performed using the newly developed orthotopic allograft mouse breast cancer model using congenic immunocompetent mice (described in the manuscript).

Doxorubicin hydrochloride (Sigma) was dissolved in ferromagnetic iron oxide nanoparticles containing stabilizing buffer and then encapsulated into PEGylated liposomes. Three weeks after transplantations of primary cells (average tumour volume 75 mm³) ferriliposomes loaded with doxorubicin were administered at a dose of 15 mg/kg (Dox+FLt, n=4). Magnets of 0.33 T (diameter 4.5 mm) were glued to the tumour by cyanoacrylate before injection and removed by acetone 24 hours after administration. Control group was treated with systemic administration of doxorubicin without any targeting (Dox, n=4). The horizontal and vertical tumour diameters were measured by digital calliper twice per week and volumes were calculated using the formula V=(a×b2)π/6 where a and b are the longer and shorter diameter of the tumour, respectively.

### Example 19

### Assessment of Ferriliposome Targeting and Internalization in Vivo by Bioluminescence

Primary MMTV-PyMT tumour cells, obtained from 14 week old MMTV-PyMT transgenic mice, were culture-expanded, suspended in 200 μl serum free Dulbecco's Modified Eagle Medium (DMEM) (Invitrogen) and injected (5×10⁵ cells) into the left inguinal mammary gland of the recipient female FVB/N mouse expressing firefly luciferase under the control of the β-actin promoter (FVB.luctg/+). For in vivo control of ferriliposomes distribution and content release, ferriliposomes were functionalized with D-luciferin (Sigma) by suspending in nanoparticles-containing stabilizing buffer (2.5 mg/ml), followed by encapsulation in PEGylated liposomes. 400 μl of ferriliposomes loaded with D-luciferin (30 mg/kg) were intraperitoneally administered to the FVB.luctg/+ mouse bearing transplanted tumour and a magnet was attached to the left inguinal mammary gland tumour. 24 hours after ferriliposomes administration magnet was detached and mice were imaged non-invasively by IVIS® Imaging System (integration time 5 minutes, IVIS® 100 Series). During the scan mice were kept under gaseous anaesthesia (5% isofluorane) and at 37° C. After whole body imaging, mice were sacrificed and the organs were harvested and imaged with an IVIS® Imaging System (2 minutes, IVIS® 100 Series). Due to the luciferase presence in all the cells, D-luciferin release and its subsequent conversion by luciferase resulted in emission of a bioluminescent signal that could be imaged with an IVIS® Imaging System.

### Example 20

### Assessment of Ferriliposome Elimination In Vivo

To clarify the pathway of ferriliposome clearance we employed transgenic mice expressing luciferase through the whole body and ferriliposomes loaded with the luciferase substrate, D-luciferin (Sigma), administered by intraperitoneal or intravenous injection and magnetic targeting. Mice were scanned non-invasively by IVIS® Imaging System (integration time 5 minutes, IVIS® 100 Series for i.p., IVIS Spectrum for i.v.). During the scan mice were kept under gaseous anaesthesia (5% isofluorane), at 37° C. The luminescence signal was clearly detected in both cases from the urinary tract of mice, providing evidence that the nanoparticles were eliminated by renal clearance, very similarly in both approaches of ferriliposome administration.

### Example 21

### Acute Toxicity Study

Female rats were treated with 500 mg/kg of iron oxide nanoparticles (n=16) and stabilizing buffer (n=8) as control. Mice were sacrificed at day 7 and 12 after injection. Blood was collected and serum separation performed by centrifugation in Li-heparin 0.6 ml flasks (Fuji Photo Film Co., Ltd. Life Science Products Division). Biochemical parameters of creatinine, urea nitrogen, alanine transaminase, aspartate transaminase, creatine phosphokinase and total bilirubin in blood were analyzed by biochemical analyzer Fujifilm DRI CHEM 3500i (Fuji Photo Film Co., Ltd. Life Science Products Division). The kidneys, spleen, liver, heart and lung tissues were collected and fixed in 10% neutral formalin. Organs were dehydrated and maintained in paraffin blocks. 5 μm paraffin sections were stained by hematoxylin and eosin for histo-pathological analysis. iron oxide nanoparticles were detected in animal tissues by Prussian blue staining with carmine (Sigma).

### Example 22

### Assay of Cysteine Cathepsin Activity

Frozen tissues of primary tumours, lungs, kidneys, pancreas and liver were disrupted in 200 μl of 0.1 M TRIS buffer (5 mM EDTA, 200 mM sodium chloride, 0.2% SDS, pH 8.5) using an Ultrathurrax (IKA, Staufen, Germany) and centrifugation at 1000 g for 10 min. Cysteine cathepsin activity was determined by hydrolysis of the general cathepsin substrate Z-Phe-Arg-4-methyl-coumarin-7-amide (Z-Phe-Arg-AMC, 25 μM; Bachem, Bubendorf, Switzerland) in 0.1 M phosphate buffer, pH 6.0, containing 1 mM EDTA, 0.1% (v/v) PEG and 1 mM dithiothreitol. Kinetics of substrate hydrolysis was monitored continuously during 10 min by spectrofluorometry at excitation and emission wavelengths of 370 and 460 nm, respectively.

### Example 23

### Quantitative Real-Time PCR

Total RNA was isolated from the tumour samples and mRNA was reverse transcribed into cDNA. RTQ-PCR was performed by detection of SYBR-green dye DNA-intercalation in the newly formed PCR-products, using the Mx3005PTM Real-Time PCR System (Agilent, Stratagene Products). The relative amount of target gene expression was normalized to the β-actin transcripts. Primers: b-actin.1: 5′-ACC CAG GCA TTG CTG ACA GG-3′ (SEQ ID No. 1); b-actin.2: 5′-GGA CAG TGA GGC CAG GAT GG-3′ (SEQ ID No. 2); Ctsb.1: 5′-TGC GTT CGG TGA GGA CAT AG-3′ (SEQ ID No. 3); Ctsb.2: 5′-CGG GCA GTT GGA CCA TTG-3′ (SEQ ID No. 4); Ctsl.1: 5′-GCA CGG CTT TTC CAT GGA-3′ (SEQ ID No. 5); Ctsl.2: 5′-CCA CCT GCC TGA ATT CCT CA-3′ (SEQ ID No. 6); Ctsx.1: 5′-TAT GCC AGC GTC ACC AGG AAC-3′ (SEQ ID No. 7); Ctsx.2: 5′-CCT CTT GAT GTT GAT TCG GTC TGC-3′ (SEQ ID No. 8); Ctsh.1: 5′-CAT GGC TGC AAA GGA GGT CT-3′ (SEQ ID No. 9); Ctsh.2: 5′-CTG TCT TCT TCC ATG ATG CCC-3′ (SEQ ID No. 10).

### Example 24

### Fluorescence Analysis of Ferriliposome Accumulation in Peritoneum Associated Lymph Nodes

For evaluation of the peritoneum clearance pathway for ferriliposomes, 200 μl of Alexa Fluor 555™-functionalized ferriliposomes were daily injected intraperitoneally into the PyMT transgenic breast cancer mouse for 3 days. Magnetic field was applied to the tumour for 12 hours immediately after each injection. 24 hours after the last injection mice were sacrificed and renal lymph nodes were resected, fixed in 10% formalin overnight, dehydrated using Shandon Tissue Processor (Shandon Citadel 1000) and moulded with paraffin (Microm EC 350 Paraffin Embedding Station). 5 μm paraffin sections were cut, mounted with anti-fade media containing DAPI (Prolong@ Gold antifade reagent with DAPI, Invitrogen) and visualized as described above.

What is claimed is:

1. A suspension comprising:
iron oxide nanoparticles or oxide ferrimagnetics with spinel structure nanoparticles
having a size between about 3 nm and 14 nm, wherein the size of 70% of the nanoparticles is smaller than 8 nm, wherein the nanoparticles are obtained by soft mechanochemical synthesis using a crystal hydrate salt of Fe; and
a biocompatible saline solution being a sterile multiplex buffer comprising:
(i) from about 50 to about 500 mM of NaCl;
(ii) from about 200 to about 2 mM of citrate buffer; and
(iii) about 100 to about 1 mM of HEPES;
wherein the buffer has a pH from about 4.0 to about 10.0.

2. The suspension according to claim 1, wherein the biocompatible saline solution comprises 20 mM of sodium citrate, 108 mM of NaCl, and 10 mM of HEPES, and wherein the pH of the biocompatible saline solution is about 7.4.

3. The suspension system of claim 1, wherein the crystal hydrate salt of Fe is FeCl₃.

4. The suspension system of claim 1, comprising from about 80 mM to about 400 mM of NaCl.

5. The suspension system of claim 1, comprising from about 100 mM to about 350 mM of NaCl.

6. The suspension system of claim 1, comprising from about 100 mM to about 10 mM of the citrate buffer.

7. The suspension system of claim 1, wherein the buffer has a pH from about 5.5 to about 9.0.

8. The suspension system of claim 1, wherein the buffer has a pH from about 6.5 to about 8.5.

9. A biocompatible aqueous colloidal system comprising iron oxide nanoparticles or oxide ferrimagnetics with spinel structure nanoparticles in accordance with claim 1.

10. A biocompatible aqueous colloidal system comprising iron oxide nanoparticles or oxide ferrimagnetics with spinel structure nanoparticles in accordance with claim 2.

11. The suspension according to claim 1 for use in diagnostic of neoplastic, neuronal and/or inflammatory diseases.

12. The suspension according to claim 2 for use in diagnostic of neoplastic, neuronal and/or inflammatory diseases.

13. The suspension according to claim 1 for use as a MRI T₁ and/or a T₂ contrast agent.

14. The suspension according to claim 2 for use as a MRI T₁ and/or a T₂ contrast agent.

15. The biocompatible aqueous colloidal system of claim 9 for use in diagnostic of neoplastic, neuronal and/or inflammatory diseases.

16. The biocompatible aqueous colloidal system of claim 10 for use in diagnostic of neoplastic, neuronal and/or inflammatory diseases.

17. The biocompatible aqueous colloidal system of claim 9 for use as a MRI T₁ and/or
a T₂ contrast agent.

18. The biocompatible aqueous colloidal system of claim 10 for use as a MRI T₁ and/or a T₂ contrast agent.

19. The suspension according to claim 1 for preparing a carrier in the form of ferriliposomes.

20. The suspension according to claim 2 for preparing a carrier in the form of ferriliposomes.

21. The biocompatible aqueous colloidal system of claim 9 for preparing a carrier in the form of ferriliposomes.

22. The biocompatible aqueous colloidal system of claim 10 for preparing a carrier in the
form of ferriliposomes.

23. A carrier in the form of ferriliposomes comprising at least one iron oxide nanoparticles or
oxide ferrimagnetics with spinel structure nanoparticles of suspension according to claim 1, at least one therapeutically active agent, at least one diagnostically active agent and at least one agent allowing targeting of the of the carrier carrier in the form of ferriliposomes, wherein:
the at least one therapeutically active agent is selected from:
a toxin,
a chemotherapeutic agent, selected from an alkylating agent, an anti-metabolite, a plant alkaloid, a taxane, a topoisomerase inhibitor, and a antineoplastic agent
a radioactive agent,
a protease inhibitor selected from a cathepsin inhibitor;
an apoptosis-inducing agent, and
an anti-inflammatory agent selected from a salicylate, a propionic acid derivative, an acetic acid derivative, an enolic acid derivative, an fenamic acid derivative, a selective COX-2 inhibitor, and a sulphonanilide;
and wherein:
the at least one diagnostically active agent is selected from a radioactive agent, a paramagnetic agent, a PET-imagable agent, an MRI-imagable agent, a fluorophore, a chromophore, a phosphorescing agent, a chemiluminescent agent, and a bioluminescent agent.

24. A carrier in the form of ferriliposomes comprising at least one iron oxide nanoparticles or
oxide ferrimagnetics with spinel structure nanoparticles of suspension according to claim 2, at least one therapeutically active agent, at least one diagnostically active agent and at least one agent allowing targeting of the of the carrier carrier in the form of ferriliposomes, wherein:
the at least one therapeutically active agent is selected from:
a toxin,
a chemotherapeutic agent, selected from an alkylating agent, an anti-metabolite, a plant alkaloid, a taxane, a topoisomerase inhibitor, and a antineoplastic agent
a radioactive agent,
a protease inhibitor selected from a cathepsin inhibitor;
an apoptosis-inducing agent, and
an anti-inflammatory agent selected from a salicylate, a propionic acid derivative, an acetic acid derivative, an enolic acid derivative, an fenamic acid derivative, a selective COX-2 inhibitor, and a sulphonanilide;
and wherein:
the at least one diagnostically active agent is selected from a radioactive agent, a paramagnetic agent, a PET-imagable agent, an MRI-imagable agent, a fluorophore, a chromophore, a phosphorescing agent, a chemiluminescent agent, and a bioluminescent agent.

25. A carrier in the form of ferriliposomes comprising at least one iron oxide nanoparticles or
oxide ferrimagnetics with spinel structure nanoparticles of biocompatible aqueous colloidal system according to claim 9, at least one therapeutically active agent, at least one diagnostically active agent and at least one agent allowing targeting of the of the carrier carrier in the form of ferriliposomes, wherein:
the at least one therapeutically active agent is selected from:
a toxin,
a chemotherapeutic agent, selected from an alkylating agent, an anti-metabolite, a plant alkaloid, a taxane, a topoisomerase inhibitor, and a antineoplastic agent
a radioactive agent,
a protease inhibitor selected from a cathepsin inhibitor
an apoptosis-inducing agent, and
an anti-inflammatory agent selected from a salicylate, a propionic acid derivative, an acetic acid derivative, an enolic acid derivative, an fenamic acid derivative, a selective COX-2 inhibitor, and a sulphonanilide and wherein:
the at least one diagnostically active agent is selected from a radioactive agent, a paramagnetic agent, a PET-imagable agent, an MRI-imagable agent, a fluorophore, a chromophore, a phosphorescing agent, a chemiluminescent agent, and a bioluminescent agent.

26. A carrier in the form of ferriliposomes comprising at least one iron oxide nanoparticles or
oxide ferrimagnetics with spinel structure nanoparticles of biocompatible aqueous colloidal system according to claim 10, at least one therapeutically active agent, at least one diagnostically active agent and at least one agent allowing targeting of the of the carrier carrier in the form of ferriliposomes, wherein:
the at least one therapeutically active agent is selected from:
a toxin,
a chemotherapeutic agent, selected from an alkylating agent, an anti-metabolite, a plant alkaloid, a taxane, a topoisomerase inhibitor, and a antineoplastic agent
a radioactive agent,
a protease inhibitor selected from a cathepsin inhibitor
an apoptosis-inducing agent, and
an anti-inflammatory agent selected from a salicylate, a propionic acid derivative, an acetic acid derivative, an enolic acid derivative, an fenamic acid derivative, a selective COX-2 inhibitor, and a sulphonanilide
and wherein:
the at least one diagnostically active agent is selected from a radioactive agent, a paramagnetic agent, a PET-imagable agent, an MRI-imagable agent, a fluorophore, a chromophore, a phosphorescing agent, a chemiluminescent agent, and a bioluminescent agent.

27. The carrier in the form of ferriliposomes according to claim 23 for use in diagnostic of
neoplastic, neuronal and/or inflammatory diseases.

28. The carrier in the form of ferriliposomes according to claim 24 for use in diagnostic of
neoplastic, neuronal and/or inflammatory diseases.

29. The carrier in the form of ferriliposomes according to claim 25 for use in diagnostic of
neoplastic, neuronal and/or inflammatory diseases.

30. The carrier in the form of ferriliposomes 26 for use in diagnostic of neoplastic, neuronal and/or inflammatory diseases.

31. The carrier in the form of ferriliposomes according to claim 25 for use as a MRI T₁
and/or a T₂ contrast agent.

32. The carrier in the form of ferriliposomes according to claim 26 for use as a MRI T₁
and/or a T₂ contrast agent.

33. A kit comprising at least one iron oxide nanoparticles or oxide ferrimagnetics with
spinel structure nanoparticles of suspension according to claim 1 and at least one magnet.

34. A kit comprising at least one iron oxide nanoparticles or oxide ferrimagnetics with
spinel structure nanoparticles of suspension according to claim 2 and at least one magnet.

35. A kit comprising at least one iron oxide nanoparticles or oxide ferrimagnetics with
spinel structure nanoparticles of biocompatible aqueous colloidal system according to claim 9 and at least one magnet.

36. A kit comprising at least one iron oxide nanoparticles or oxide ferrimagnetics with
spinel structure nanoparticles of biocompatible aqueous colloidal system according to claim 10 and at least one magnet.
